(19) United States
(12) Patent Application Publication
(10) Pub. No.: US 2024/0369459 A1
(43) Pub. Date: Nov. 7, 2024

(54) METHODS AND COMPOSITIONS FOR DECROSSLINKING BIOLOGICAL SAMPLES

(71) Applicant: Promega Corporation, Madison, WI (US)

(72) Inventors: Kevin Mayer, Madison, WI (US); Hui Wang, Madison, WI (US); Douglas Horejsh, Madison, WI (US); Marjeta Urh, Madison, WI (US); Wenhui Zhou, Madison, WI (US)

(21) Appl. No.: 18/654,908

(22) Filed: May 3, 2024

Related U.S. Application Data
(60) Provisional application No. 63/500,184, filed on May 4, 2023.

(51) Int. Cl. G01N 1/30; C07C 211/03; C07D 261/02; C07D 265/02; C07F 9/38; C12N 9/58

(52) U.S. Cl. CPC G01N 1/30; C07C 211/03; C07D 261/02; C07D 265/02; C07F 9/3834; C12N 9/58; C12Y 304/21064

(57) ABSTRACT

Provided herein are methods, compositions, and kits for decrosslinking formaldehyde cross-linked biological samples such as formalin-fixed, paraffin-embedded (FFPE) tissue samples.

### CROSS-REFERENCE TO RELATED APPLICATIONS

This application claims priority to and the benefit of U.S. Provisional Patent Application No. 63/500,184, filed on May 4, 2023, which is incorporated herein by reference in its entirety.

### FIELD

Provided herein are methods, compositions, and kits for decrosslinking formaldehyde cross-linked biological samples such as formalin-fixed, paraffin-embedded (FFPE) tissue samples.

### BACKGROUND

Formalin Fixed Paraffin Embedded (FFPE) tissue specimens are precious clinical samples that contain a great deal of patient information. When such samples are prepared, formalin acts as a preservative by creating extensive molecular crosslinks. Before nucleic acids or other biomolecules can be extracted and used in downstream assays, the crosslinks must be reversed, which requires significant preprocessing time. Typically, after deparaffinization and a short Proteinase K digestion, tissues are decrosslinked for long periods (hours to days) at high temperatures (60-90° C.); the industry standard is 4 hours at 80° C. While this decrosslinking step is necessary, it also can contribute to extraction failure and poor sample quality, which poses a significant challenge for clinical and research applications.

### SUMMARY

In one aspect, disclosed herein is a method of decrosslinking a formaldehyde cross-linked biological sample, comprising a step of simultaneously contacting the sample with a protease and a decrosslinking catalyst, wherein the decrosslinking catalyst is a compound of formula (I), formula (II), or formula (III);
wherein the compound of formula (I) is:

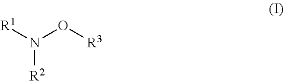

or a salt thereof,
wherein:
R¹ is selected from H and C₁-C₆ alkyl;
R² is selected from H, C₁-C₆ alkyl, aryl, heteroaryl, aryl-C₁-C₄-alkyl, and heteroaryl-C₁-C₄-alkyl;
R³ is selected from H, C₁-C₆ alkyl, and —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or more substituents independently selected from halo, C₁-C₄ alkyl, hydroxy, C₁-C₄ alkoxy, —NR^{a1}R^{b1}, —C(O)NR^{c1}R^{d1}, —COOR^{e1}, and —SO₂NR^{f1}R^{g1};
wherein each alkyl is unsubstituted or substituted with 1 or more substituents independently selected from halo, hydroxy, C₁-C₄ alkoxy, thiol, C₁-C₄ alkylthio, —NR^{a1}R^{b1}, —C(O)NR^{c1}R^{d1}, —COOR^{e1}, —SO₂NR^{f1}R^{g1}- , —ONR^{h1}Rⁱ¹, —NR^{j1}OR^{k1}, —PO₃H₂, —SO₃H, optionally substituted C₃-C₆ cycloalkyl, and optionally substituted 3- to 6-membered heterocyclyl; and
wherein R^{a1}, R^{b1}, R^{c1}, R^{d1}, R^{e1}, R^{f1}, R^{g1}, R^{h1}, Rⁱ¹, R^{j1}, and R^{k1} are each independently selected from H, C₁-C₄ alkyl, C₁-C₄ hydroxyalkyl, and C₁-C₄ carboxyalkyl;
wherein R¹ and R², together with the nitrogen atom to which they are attached, are optionally taken together to form an optionally substituted 4- to 8-membered ring; and
wherein R² and R³, together with the atoms to which they are attached, are taken together to form an optionally substituted 4- to 8-membered ring;
   wherein the compound of formula (II) is:

A—NR^{x}R^{y} (II)
or a salt thereof, wherein:
A is selected from H, C₁-C₆-alkyl, aryl, heteroaryl, C₃-C₆-cycloalkyl, aryl-C₁-C₄-alkyl, heteroaryl-C₁-C₄-alkyl, and —Q—R⁵;
Q is selected from —NR^{a2}—, —NR^{b2}CO—, —SO₂—, —SO₂NR^{c2}—, and —NR^{d2}COCONR^{e2}NR^{f2}—;
R⁵ is selected from H, C₁-C₆-alkyl, aryl, heteroaryl, and C₃-C₆-cycloalkyl;
R^{a2}, R^{b2}, R^{c2}, R^{d2}, R^{e2}, and R^{f2} are each independently selected from H and C₁-C₆ alkyl;
R^{x} and R^{y} are each independently selected from H and C₁-C₆ alkyl; or R^{x} and R^{y}, together with the nitrogen atom to which they are attached, form an optionally substituted 4- to 8-membered ring;
wherein each aryl or heteroaryl is unsubstituted or substituted independently selected from halo, hydroxyl, C₁-C₄ alkyl, C₁-C₄ alkoxy, C₁-C₄ hydroxyalkyl, —P(O)(OH)2, —B(OH)2, —COOR^{g2}, —CONR^{h2}Rⁱ², —SO₂NR^{j2}R^{k2}, and —SO₂OR^{l2}; and
wherein each alkyl or cycloalkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOR^{g2}, —PO₃H₂, —SO₂OR^{l2}, and —NR^{m2}Rⁿ²;
wherein each R^{g2}, R^{h2}, Rⁱ², R^{j2}, R^{k2}, R^{l2}, R^{m2}, and Rⁿ² is independently selected from H and methyl;
   wherein the compound of formula (III) is:

B—X (III)
or a salt thereof, wherein:
B is aryl, heteroaryl, C₁-C₆ alkyl, aryl-C₁-C₄-alkyl-, aryl-C₂-C₄-alkoxy-, heteroaryl-C₁-C₄-alkyl-, and heteroaryl-C₂-C₄-alkoxy-; and
X is selected from —COOH, —PO₃H₂, —B(OH)₂, and —SO₃H;
wherein each aryl and heteroaryl is independently unsubstituted or substituted with 1 or more substituents independently selected from halo, C₁-C₄ alkyl, C₁-C₄ alkoxy, hydroxy, C₁-C₄ hydroxyalkyl, —NR^{a3}R^{b3}, —COOR^{c3}, —SO₃R^{d3}, —PO₃H₂, and —B(OH)₂;
wherein each alkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOH, —PO₃H₂ and —SO₃H; and
wherein R^{a3}, R^{b3}, R^{c3}, and R^{d3} are each independently selected from H, —CH₃, and —CH₂CH₃.

In some embodiments, the decrosslinking catalyst is a compound of formula (I), or a salt thereof.

In some embodiments: R¹ is H; R² is selected from H, C₁-C₆ alkyl, —CH₂-aryl, and —CH₂-heteroaryl; and R³ is selected from H, C₁-C₆ alkyl, —X—R⁴, wherein X is selected from —C(O)— and —SO₂—, and R⁴ is selected from C₁-C₆-alkyl, aryl, and heteroaryl; wherein each aryl and heteroaryl is independently unsubstituted or substituted with 1 or 2 substituents independently selected from halo, hydroxy and C₁-C₄ alkoxy; and wherein each alkyl is independently unsubstituted or substituted with 1 substituent selected from hydroxy, C₁-C₄ alkoxy, —NR^{a1}R^{b1}, —COOR^{c1}, —ONR^{h1}Rⁱ¹, and —NR^{j1}OR^{k1}, wherein R^{a1}, R^{b1}, R^{e1}, R^{h1}, Rⁱ¹, R^{j1}, and R^{k1} are each independently selected from H and —CH₃.

In some embodiments: R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and R³ is H.

In some embodiments: R¹ is H; and R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo.

In some embodiments, the compound of formula (I) is selected from:

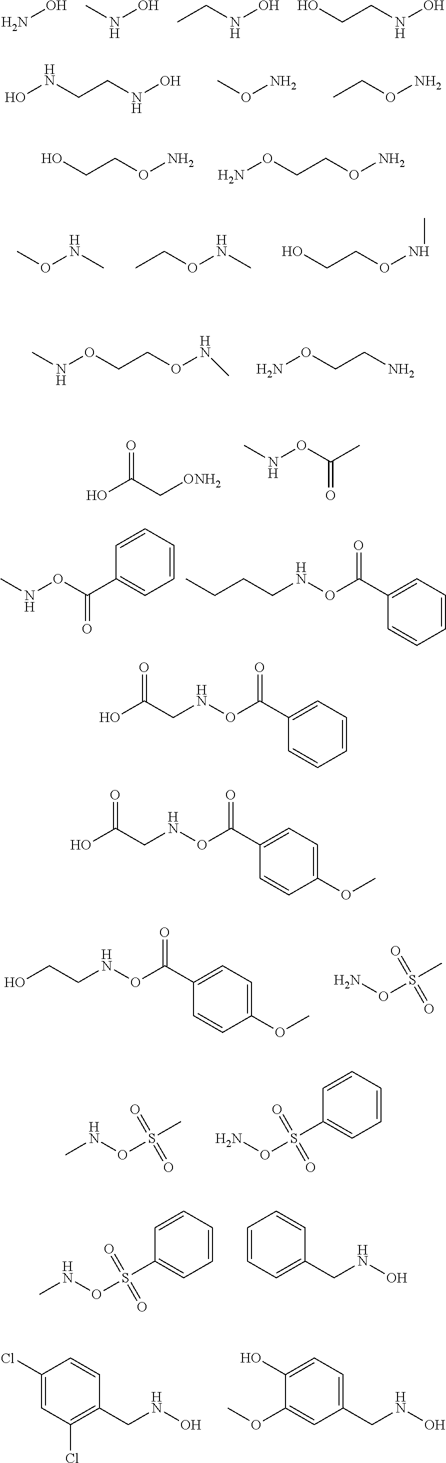

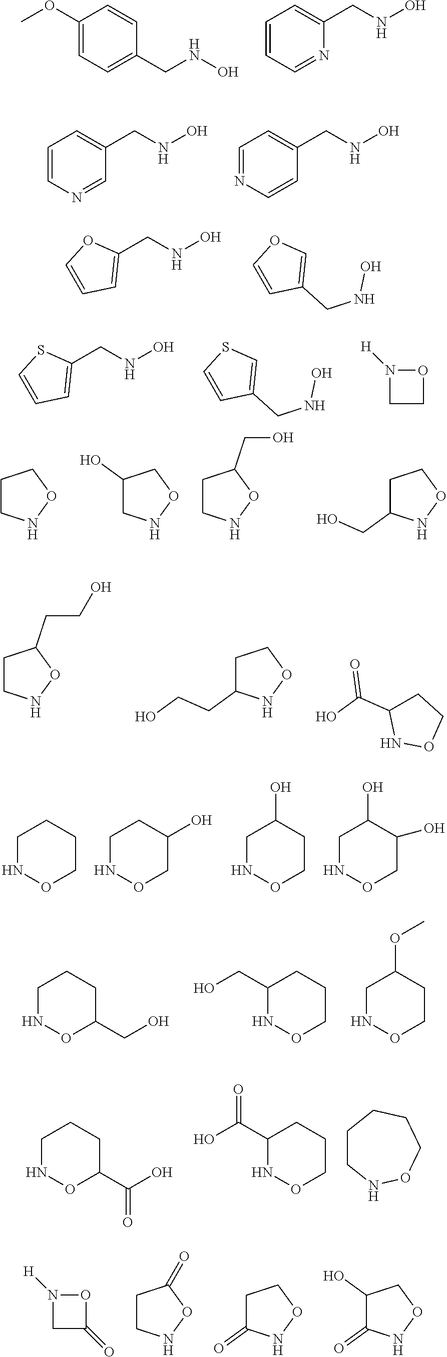

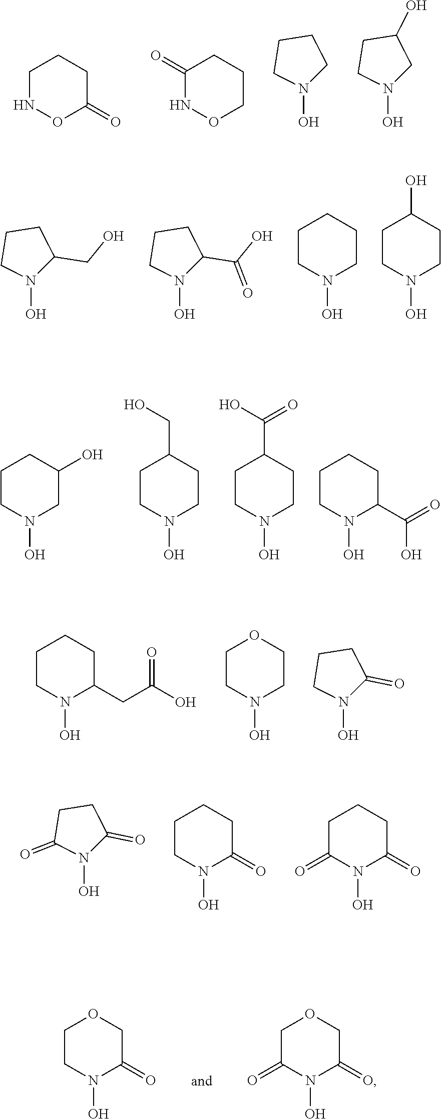

and salts thereof.

In some embodiments, the decrosslinking catalyst is a compound of formula (II), or a salt thereof.

In some embodiments, R^{x} is H, and R^{y} is selected from H and —CH₃. In some embodiments, A is selected from phenyl, monocyclic heteroaryl, and C₅-C₆-cycloalkyl, each of which is unsubstituted or substituted with 1 or 2 substituents independently selected from C₁-C₄ alkyl, C₁-C₄ alkoxy, —P(O)(OH)₂, —B(OH)₂, and —COOH.

In some embodiments: A is selected from C₁-C₆-alkyl and —Q—R⁵; Q is selected from —NR^{a2}, —NR^{b2}CO—, —SO₂—, and —NR^{d2}COCONR^{e2}NR^{f2}—; and R⁵ is H, C₁-C₆-alkyl, phenyl, or a monocyclic heteroaryl; wherein each alkyl is unsubstituted or substituted with 1 or 2 substituents independently selected from hydroxy, —P(O)(OH)₂, —COOR^{g2}, and —NR^{m2}Rⁿ²; and wherein R^{a2}, R^{b2}, R^{d2}, R^{e2}, R^{f2}, R^{g2}, R^{m2}, and Rⁿ² are each H.

In some embodiments: R^{x} and R^{y}, together with the nitrogen atom to which they are attached, form a 6-membered ring selected from morpholine, thiomorpholine, selenomorpholine, and piperazine, each of which is unsubstituted or substituted with 1 or 2 oxo groups; A is selected from H, —CH₃, —CH₂CH₃, —CH₂COOH, —CH₂CH₂OH, and —Q—R⁵; Q is —NR^{a2}— or —NR^{b2}CO—, wherein R^{a2} and R^{b2} are each H; and R⁵ is H or —CH₃.

In some embodiments, the compound of formula (II) is selected from:

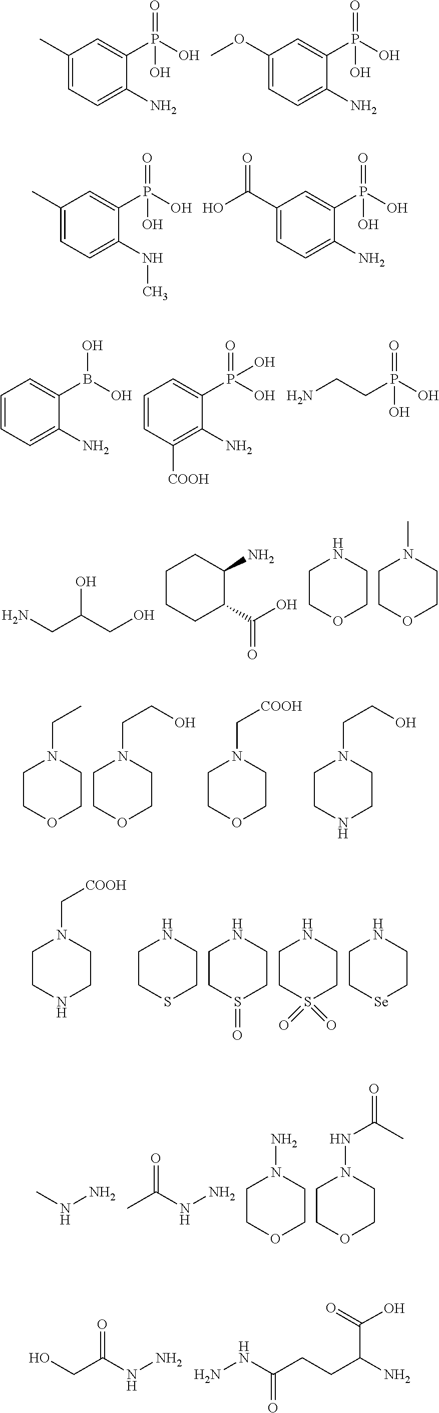

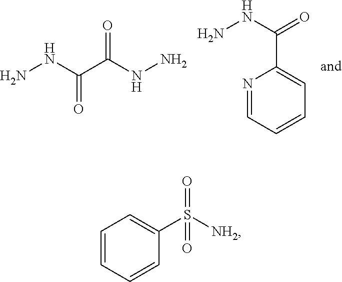 [SMILES: NNC(=O)C(=O)NN ; NNC(=O)c1ccccn1 ; NS(=O)(=O)c1ccccc1]

and salts thereof.

In some embodiments, the decrosslinking catalyst is a compound of formula (III), or a salt thereof.

In some embodiments, B is aryl or heteroaryl, each of which is unsubstituted or substituted with 1 or 2 substituents independently selected from halo, methyl, methoxy, —COOH, and —PO₃H₂; and X is selected from —COOH, —PO₃H₂, and —SO₃H.

In some embodiments, B is phenyl or a monocyclic heteroaryl having one heteroatom selected from N, O, and S; each of which is unsubstituted or substituted with 1 substituent selected from methyl, methoxy, fluoro, chloro, —COOH, and —PO₃H₂; and X is selected from —COOH, —PO₃H₂, and —SO₃H.

In some embodiments, the compound of formula (III) is selected from:

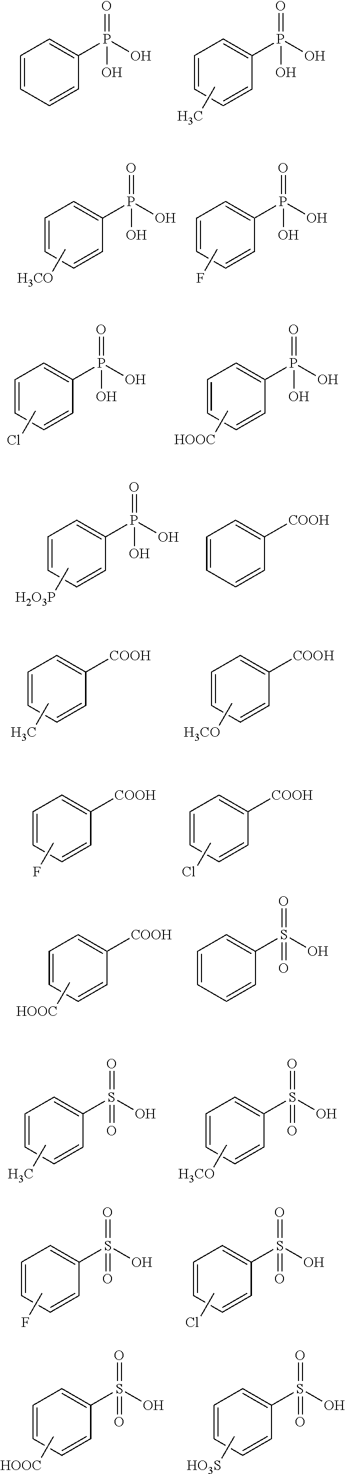

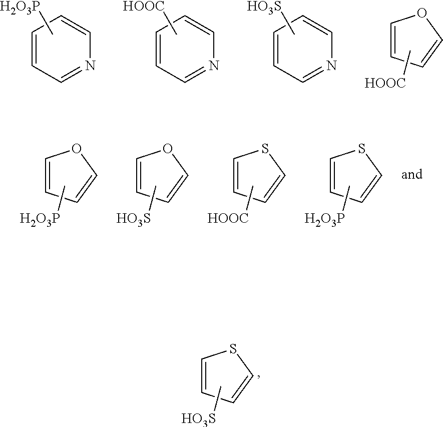

and salts thereof.

In some embodiments, the decrosslinking catalyst is selected from:

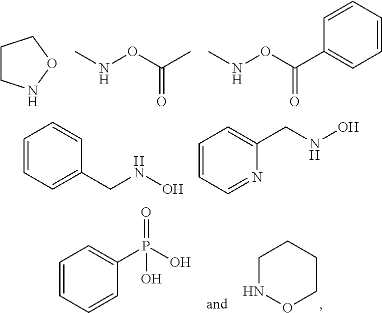

and salts thereof.

In some embodiments, the decrosslinking catalyst is in the form of a salt. In some embodiments, the decrosslinking catalyst is in the form of a hydrochloric acid salt.

In some embodiments, the method comprises contacting the sample with effective amounts of at least two different decrosslinking catalysts, or salts thereof. In some embodiments, the at least two different decrosslinking catalysts, or salts thereof, are added to the sample simultaneously. In some embodiments, the at least two different decrosslinking catalysts, or salts thereof, are added to the sample sequentially.

In some embodiments, the sample is a formalin-fixed paraffin embedded tissue sample. In some embodiments, the method further comprises a step of deparaffinizing the sample prior to contacting the sample with the protease and the decrosslinking catalyst. In some embodiments, the protease is proteinase K.

In some embodiments, the contacting step is conducted for about 5 minutes to about 120 minutes. In some embodiments, the contacting step is conducted for about 20 minutes to about 40 minutes. In some embodiments, the contacting step is conducted at a temperature of about 20° C. to about 100° C. In some embodiments, the contacting step is conducted at a temperature of about 50° C. to about 85° C.

In some embodiments, the method comprises contacting the sample with a solution comprising the decrosslinking catalyst and the protease. In some embodiments, upon contacting the sample with the solution, the resulting solution has a pH of about 4.0 to about 8.5. In some embodiments, the resulting solution has a pH of about 4.5 to about 6.5.

In some embodiments, the solution comprises the decrosslinking catalyst at a concentration of about 1 mM to about 100 mM. In some embodiments, the solution comprises the decrosslinking catalyst at a concentration of about 10 mM to about 50 mM.

In some embodiments, the protease and/or a decrosslinking catalyst are added in a buffered solution. In some embodiments, the buffered solution comprises tris(hydroxymethyl)aminomethane (Tris), 2-(N-morpholino)ethanesulfonic acid (MES), 4-(2-hydroxyethyl)-1-piperazineethanesulfonic acid (HEPES), phosphate-buffered saline, glycine, or citrate.

In some embodiments, the method comprises contacting the sample with a first solution comprising the protease and a second solution comprising the decrosslinking catalyst. In some embodiments, the pH of the resulting solution after the contacting step is about 4.0 to about 8.5. In some embodiments, the pH of the resulting solution after the contacting step is about 4.5 to about 6.5. In some embodiments, the second solution comprises the decrosslinking catalyst at a concentration of about 1 mM to about 100 mM. In some embodiments, the second solution comprises the decrosslinking catalyst at a concentration of about 5 mM to about 50 mM.

In some embodiments, the method further comprises extracting one or more components from the sample after the contacting step. In some embodiments, the one or more components are selected from nucleic acids and proteins. In some embodiments, the one or more components are nucleic acids, and the method further comprises a step of detecting and/or amplifying one or more nucleic acids. In some embodiments, the method further comprises a step selected from dye binding, absorbance, and enzymatic digestion.

In another aspect, disclosed herein is a composition comprising a protease and a decrosslinking catalyst, wherein the decrosslinking catalyst is a compound of formula (I), formula (II), or formula (III):

wherein the compound of formula (I) is:

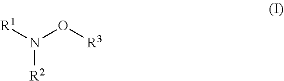

or a salt thereof,
wherein:
R¹ is selected from H and C₁-C₆ alkyl;
R² is selected from H, C₁-C₆ alkyl, aryl, heteroaryl, aryl-C₁-C₄-alkyl, and heteroaryl-C₁-C₄-alkyl;
R³ is selected from H, C₁-C₆ alkyl, and —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or more substituents independently selected from halo, C₁-C₄ alkyl, hydroxy, C₁-C₄ alkoxy, —NR^{a1}R^{b1}, —C(O)NR^{c1}R^{d1}, —COOR^{e1}, and —SO₂NR^{f1}R^{g1};
wherein each alkyl is unsubstituted or substituted with 1 or more substituents independently selected from halo, hydroxy, C₁-C₄ alkoxy, thiol, C₁-C₄ alkylthio, —NR^{a1}R^{b1}, —C(O)NR^{c1}R^{d1}, —COOR^{e1}, —SO₂NR^{f1}R^{g1}, —ONR^{h1}Rⁱ¹, —NR^{j1}OR^{k1}, —PO₃H₂, —SO₃H, optionally substituted C₃-C₆ cycloalkyl, and optionally substituted 3- to 6-membered heterocyclyl; and
wherein R^{a1}, R^{b1}, R^{c1}, R^{d1}, R^{e1}, R^{f1}, R^{g1}, R^{h1}, Rⁱ¹, R^{j1}, and R^{k1} are each independently selected from H, C₁-C₄ alkyl, C₁-C₄ hydroxyalkyl, and C₁-C₄ carboxyalkyl;
wherein R¹ and R², together with the nitrogen atom to which they are attached, are optionally taken together to form an optionally substituted 4- to 8-membered ring; and
wherein R² and R³, together with the atoms to which they are attached, are taken together to form an optionally substituted 4- to 8-membered ring;
   wherein the compound of formula (II) is:

A—NR^{x}R^{y} (II)
or a salt thereof, wherein:
A is selected from H, C₁-C₆-alkyl, aryl, heteroaryl, C₃-C₆-cycloalkyl, aryl-C₁-C₄-alkyl, heteroaryl-C₁-C₄-alkyl, and —Q—R⁵;
Q is selected from —NR^{a2}—, —NR^{b2}CO—, —SO₂—, —SO₂NR^{c2}—, and —NR^{d2}COCONR^{e2}NR^{f2}—;
R⁵ is selected from H, C₁-C₆-alkyl, aryl, heteroaryl, and C₃-C₆-cycloalkyl;
R^{a2}, R^{b2}, R^{e2}, R^{d2}, R^{e2}, and R^{f2} are each independently selected from H and C₁-C₆ alkyl;
R^{x} and R^{y} are each independently selected from H and C₁-C₆ alkyl; or R^{x} and R^{y}, together with the nitrogen atom to which they are attached, form an optionally substituted 4- to 8-membered ring;
wherein each aryl or heteroaryl is unsubstituted or substituted independently selected from halo, hydroxyl, C₁-C₄ alkyl, C₁-C₄ alkoxy, C₁-C₄ hydroxyalkyl, —P(O)(OH)₂, —B(OH)₂, —COOR^{g2}, —CONR^{h2}Rⁱ², —SO₂NR^{j2}R^{k2}, and —SO₂OR^{l2}; and
wherein each alkyl or cycloalkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOR^{g2}, —PO₃H₂, —SO₂OR^{l2}, and —NR^{m2}Rⁿ²;
wherein each R^{g2}, R^{h2}, Rⁱ², R^{j2}, R^{k2}, R^{l2}, R^{m2}, and Rⁿ² is independently selected from H and methyl;
   wherein the compound of formula (III) is:

B—X (III)
or a salt thereof, wherein:
B is aryl, heteroaryl, C₁-C₆ alkyl, aryl-C₁-C₄-alkyl-, aryl-C₂-C₄-alkoxy-, heteroaryl-C₁-C₄-alkyl-, and heteroaryl-C₂-C₄-alkoxy-; and
X is selected from —COOH, —PO₃H₂, —B(OH)₂, and —SO₃H;
wherein each aryl and heteroaryl is independently unsubstituted or substituted with 1 or more substituents independently selected from halo, C₁-C₄ alkyl, C₁-C₄ alkoxy, hydroxy, C₁-C₄ hydroxyalkyl, —NR^{a3}R^{b3}, —COOR^{c3}, —SO₃R^{d3}, —PO₃H₂, and —B(OH)₂;
wherein each alkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOH, —PO₃H₂ and —SO₃H; and
wherein R^{a3}, R^{b3}, R^{c3}, and R^{d3} are each independently selected from H, —CH₃, and —CH₂CH₃.

In some embodiments, the decrosslinking catalyst is a compound of formula (I), or a salt thereof.

In some embodiments: R¹ is H; R² is selected from H, C₁-C₆ alkyl, —CH₂-aryl, and —CH₂-heteroaryl; and R³ is selected from H, C₁-C₆ alkyl, —X—R⁴, wherein X is selected from —C(O)— and —SO₂—, and R⁴ is selected from C₁-C₆-alkyl, aryl, and heteroaryl; wherein each aryl and heteroaryl is independently unsubstituted or substituted with 1 or 2 substituents independently selected from halo, hydroxy and C₁-C₄ alkoxy; and wherein each alkyl is independently unsubstituted or substituted with 1 substituent selected from hydroxy, C₁-C₄ alkoxy, —NR^{a1}R^{b1}, —COOR^{e1}, —ONR^{h1}Rⁱ¹, and —NR^{j1}OR^{k1}; wherein R^{a1}, R^{b1}, R^{e1}, R^{h1}, Rⁱ¹, R^{j1}, and R^{k1} are each independently selected from H and —CH₃.

In some embodiments: R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and R³ is H.

In some embodiments: R¹ is H; and R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo.

In some embodiments, the compound of formula (I) is selected from:

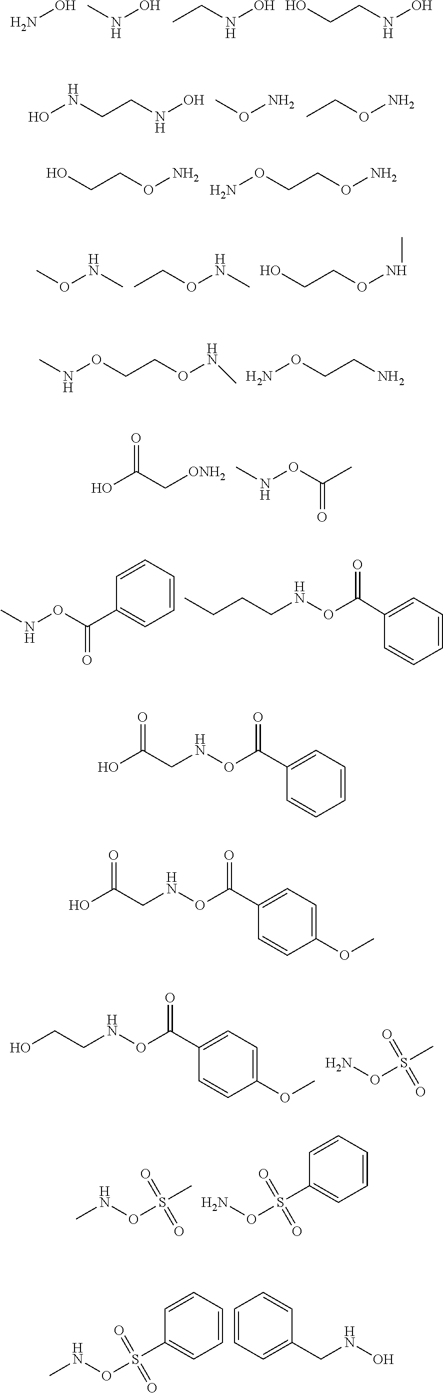

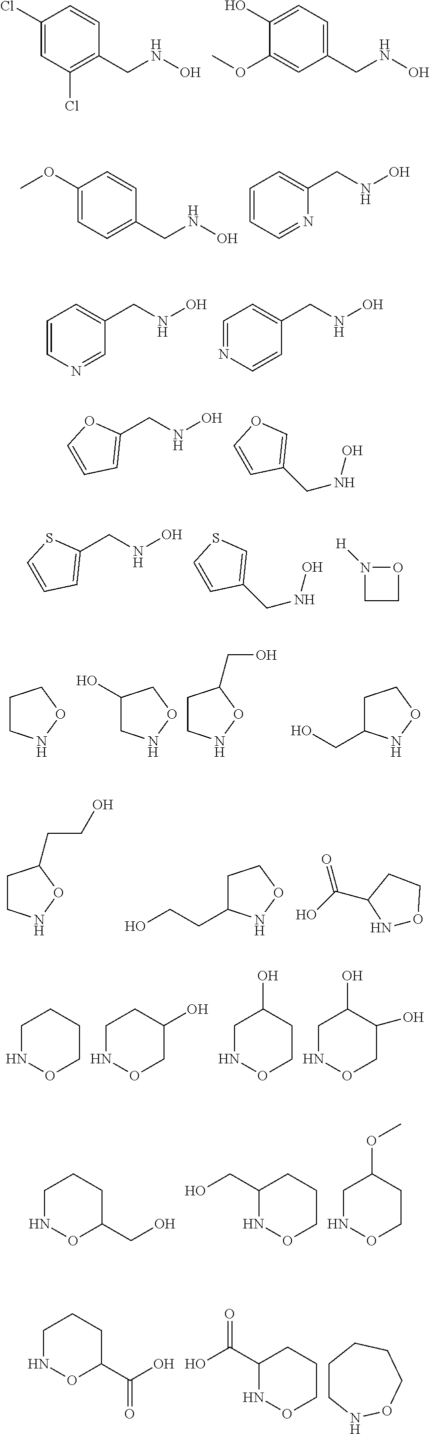

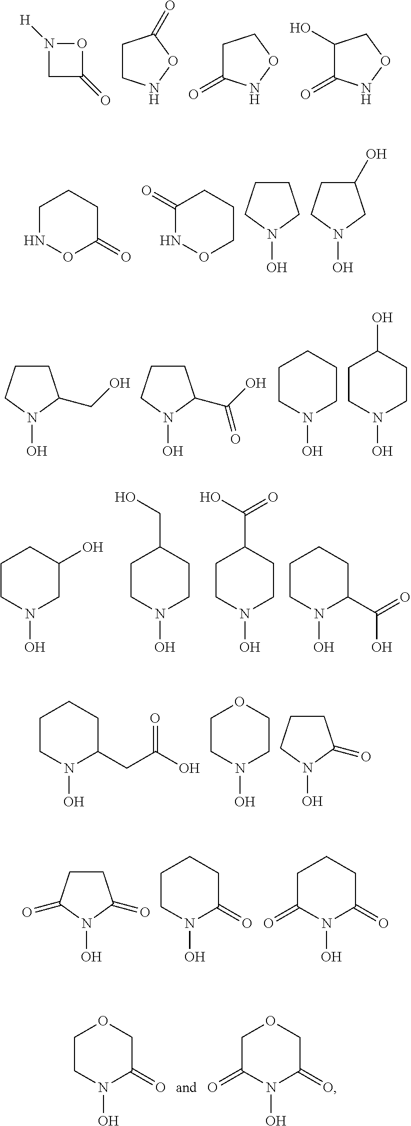

and salts thereof.

In some embodiments, the decrosslinking catalyst is a compound of formula (II), or a salt thereof. In some embodiments, R^{x} is H, and R^{y} is selected from H and —CH₃. In some embodiments, A is selected from phenyl, monocyclic heteroaryl, and C₅-C₆-cycloalkyl, each of which is unsubstituted or substituted with 1 or 2 substituents independently selected from C₁-C₄ alkyl, C₁-C₄ alkoxy, —P(O)(OH)₂, —B(OH)₂, and —COOH. In some embodiments: A is selected from C₁-C₆ alkyl and —Q—R⁵; Q is selected from —NR^{a2}—, —NR^{b2}CO—, —SO₂—, and —NR^{d2}COCONR^{e2}NR^{f2}—; and R⁵ is H, C₁-C₆-alkyl, phenyl, or a monocyclic heteroaryl; wherein each alkyl is unsubstituted or substituted with 1 or 2 substituents independently selected from hydroxy, —P(O)(OH)₂, —COOR^{g2}, and —NR^{m2}Rⁿ²; and wherein R^{a2}, R^{b2}, R^{d2}, R^{e2}, R^{f2}, R^{g2}, R^{m2}, and Rⁿ² are each H.

In some embodiments: R^{x} and R^{y}, together with the nitrogen atom to which they are attached, form a 6-membered ring selected from morpholine, thiomorpholine, selenomorpholine, and piperazine, each of which is unsubstituted or substituted with 1 or 2 oxo groups; A is selected from H, —CH₃, —CH₂CH₃, —CH₂COOH, —CH₂CH₂OH, and —Q—R⁵; Q is —NR^{a2}— or —NR^{b2}CO—, wherein R^{a2} and R^{b2} are each H; and R⁵ is H or —CH₃.

In some embodiments the compound of formula (II) is selected from:

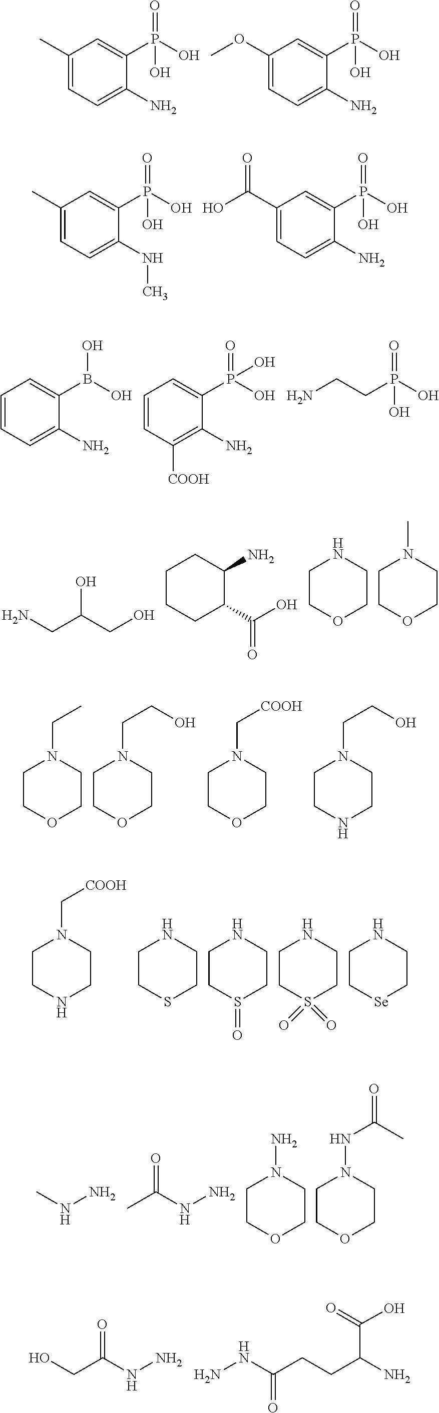

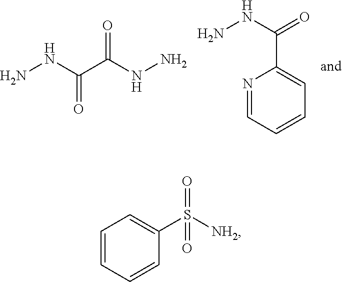 [SMILES: NNC(=O)C(=O)NN ; NNC(=O)c1ccccn1 ; NS(=O)(=O)c1ccccc1]

and salts thereof.

In some embodiments the decrosslinking catalyst is a compound of formula (III), or a salt thereof. In some embodiments: B is aryl or heteroaryl, each of which is unsubstituted or substituted with 1 or 2 substituents independently selected from halo, methyl, methoxy, —COOH, and —PO₃H₂; and X is selected from —COOH, —PO₃H₂, and —SO₃H. In some embodiments: B is phenyl or a monocyclic heteroaryl having one heteroatom selected from N, O, and S; each of which is unsubstituted or substituted with 1 substituent selected from methyl, methoxy, fluoro, chloro, —COOH, and —PO₃H₂; and X is selected from —COOH, —PO₃H₂, and —SO₃H.

In some embodiments, the compound of formula (III) is selected from:

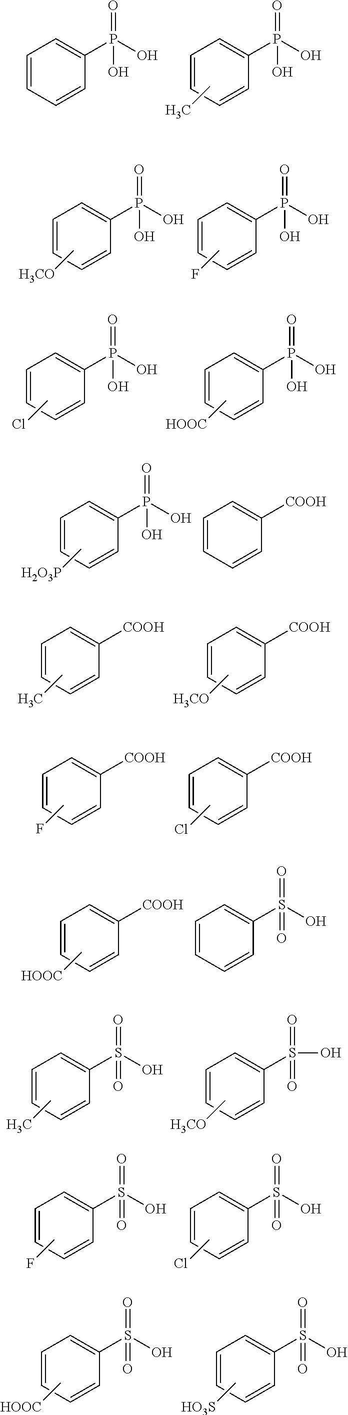

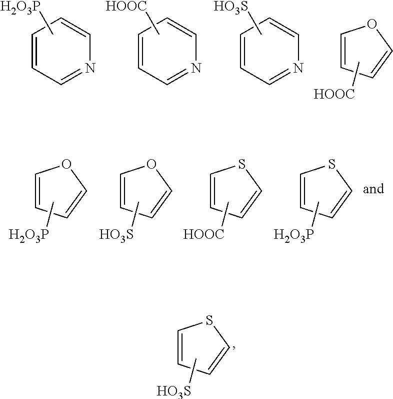

and salts thereof.

In some embodiments, the decrosslinking catalyst is selected from:

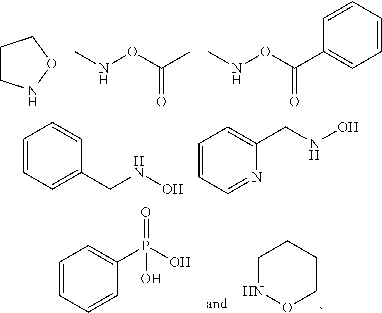

and salts thereof.

In some embodiments, the decrosslinking catalyst is in the form of a salt. In some embodiments, the decrosslinking catalyst is in the form of a hydrochloric acid salt.

In some embodiments, the protease is proteinase K.

In some embodiments, the composition comprises a solution of the decrosslinking catalyst and the protease. In some embodiments, the solution has a pH of about 4.0 to about 8.5 upon adding the solution of the decrosslinking catalyst and the protease to a cross-linked biological sample. In some embodiments, the solution has a pH of about 4.5 to about 6.5 upon adding the solution of the decrosslinking catalyst and the protease to a cross-linked biological sample. In some embodiments, the solution comprises the decrosslinking catalyst at a concentration of about 1 mM to about 100 mM. In some embodiments, the solution comprises the decrosslinking catalyst at a concentration of about 10 mM to about 50 mM.

In some embodiments, the composition comprises at least two different decrosslinking catalysts, or salts thereof.

In some embodiments, the composition further comprises a buffer. In some embodiments, the buffer is tris(hydroxymethyl)aminomethane.

In some embodiments, the composition further comprises a formaldehyde cross-linked biological sample. In some embodiments, the sample is a formalin-fixed paraffin embedded tissue sample.

In another aspect, disclosed herein is a kit for decrosslinking a formaldehyde cross-linked biological sample, the kit comprising: (i) a decrosslinking catalyst, wherein the decrosslinking catalyst is a compound of formula (I), formula (II), or formula (III); and (ii) instructions for decrosslinking a formaldehyde cross-linked biological sample, instructing a user to simultaneously contact the sample with the decrosslinking catalyst and a protease; wherein the compound of formula (I) is:

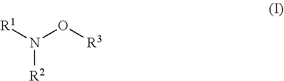

or a salt thereof,
wherein:
R¹ is selected from H and C₁-C₆ alkyl;
R² is selected from H, C₁-C₆ alkyl, aryl, heteroaryl, aryl-C₁-C₄-alkyl, and heteroaryl-C₁-C₄-alkyl;
R³ is selected from H, C₁-C₆ alkyl, and —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or more substituents independently selected from halo, C₁-C₄ alkyl, hydroxy, C₁-C₄ alkoxy, —NR^{a1}R^{b1}, —C(O)NR^{c1}R^{d1}, —COOR^{e1}, and —SO₂NR^{f1}R^{g1};
wherein each alkyl is unsubstituted or substituted with 1 or more substituents independently selected from halo, hydroxy, C₁-C₄ alkoxy, thiol, C₁-C₄ alkylthio, —NR^{a1}R^{b1}, —C(O)NR^{c1}R^{d1}, —COOR^{e1}, —SO₂NR^{f1}R^{g1}, —ONR^{h1}Rⁱ¹, —NR^{j1}OR^{k1}, —PO₃H₂, —SO₃H, optionally substituted C₃-C₆ cycloalkyl, and optionally substituted 3- to 6-membered heterocyclyl; and
wherein R^{a1}, R^{b1}, R^{c1}, R^{d1}, R^{e1}, R^{f1}, R^{g1}, R^{h1}, Rⁱ¹, R^{j1}, and R^{k1} are each independently selected from H, C₁-C₄ alkyl, C₁-C₄ hydroxyalkyl, and C₁-C₄ carboxyalkyl;
wherein R¹ and R², together with the nitrogen atom to which they are attached, are optionally taken together to form an optionally substituted 4- to 8-membered ring; and
wherein R² and R³, together with the atoms to which they are attached, are taken together to form an optionally substituted 4- to 8-membered ring:
   wherein the compound of formula (II) is:

A—NR^{x}R^{y} (II)
or a salt thereof, wherein:
A is selected from H, C₁-C₆-alkyl, aryl, heteroaryl, C₃-C₆-cycloalkyl, aryl-C₁-C₄-alkyl, heteroaryl-C₁-C₄-alkyl, and —Q—R⁵;
Q is selected from —NR^{a2}—, —NR^{b2}CO—, —SO₂—, —SO₂NR^{c2}—, and —NR^{d2}COCONR^{e2}NR^{f2}—;
R⁵ is selected from H, C₁-C₆-alkyl, aryl, heteroaryl, and C₃-C₆-cycloalkyl;
R^{a2}, R^{b2}, R^{c2}, R^{d2}, R^{e2}, and R^{f2} are each independently selected from H and C₁-C₆ alkyl;
R^{x} and R^{y} are each independently selected from H and C₁-C₆ alkyl; or R^{x} and R^{y}, together with the nitrogen atom to which they are attached, form an optionally substituted 4- to 8-membered ring;
wherein each aryl or heteroaryl is unsubstituted or substituted independently selected from halo, hydroxyl, C₁-C₄ alkyl, C₁-C₄ alkoxy, C₁-C₄ hydroxyalkyl, —P(O)(OH)₂, —B(OH)₂, —COOR^{g2}, —CONR^{h2}Rⁱ², —SO₂NR^{j2}R^{k2}, and —SO₂OR^{l2}; and
wherein each alkyl or cycloalkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOR^{g2}, —PO₃H₂, —SO₂OR^{l2}, and —NR^{m2}Rⁿ²;
wherein each R^{g2}, R^{h2}, Rⁱ², R^{j2}, R^{k2}, R^{l2}, R^{m2}, and Rⁿ² is independently selected from H and methyl;
   wherein the compound of formula (III) is:

B—X (III)
or a salt thereof, wherein:
B is aryl, heteroaryl, C₁-C₆ alkyl, aryl-C₁-C₄-alkyl-, aryl-C₂-C₄-alkoxy-, heteroaryl-C₁-C₄-alkyl-, and heteroaryl-C₂-C₄-alkoxy-; and
X is selected from —COOH, —PO₃H₂, —B(OH)₂, and —SO₃H;
wherein each aryl and heteroaryl is independently unsubstituted or substituted with 1 or more substituents independently selected from halo, C₁-C₄ alkyl, C₁-C₄ alkoxy, hydroxy, C₁-C₄ hydroxyalkyl, —NR^{a3}R^{b3}, —COOR^{c3}, —SO₃R^{d3}, —PO₃H₂, and —B(OH)₂;
wherein each alkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOH, —PO₃H₂ and —SO₃H; and
wherein R^{a3}, R^{b3}, R^{c3}, and R^{d3} are each independently selected from H, —CH₃, and —CH₂CH₃.

In some embodiments, the kit further comprises a protease. In some embodiments, the protease is proteinase K.

In some embodiments, the kit comprises at least two different decrosslinking catalysts, or salts thereof.

In some embodiments, the kit further comprises at least one component selected from a buffer, a salt, and packaging material.

In another aspect, disclosed herein is a method of decrosslinking a formaldehyde cross-linked biological sample, comprising a step of simultaneously contacting the sample with a protease and at least two different decrosslinking catalysts, wherein each decrosslinking catalyst is a compound of formula (I), formula (II), or formula (III), as defined herein.

In another aspect, disclosed herein is a composition comprising a protease and at least two different decrosslinking catalysts, wherein each decrosslinking catalyst is a compound of formula (I), formula (II), or formula (III), as defined herein.

In another aspect, disclosed herein is a kit for decrosslinking a formaldehyde cross-linked biological sample, the kit comprising: (i) at least two different decrosslinking catalysts, wherein each decrosslinking catalyst is a compound of formula (I), formula (II), or formula (III), as defined herein; and (ii) instructions for decrosslinking a formaldehyde cross-linked biological sample, instructing a user to simultaneously contact the sample with the decrosslinking catalyst and a protease.

In another aspect, disclosed herein is a kit for decrosslinking a formaldehyde cross-linked biological sample, the kit comprising: (i) at least two different decrosslinking catalysts, wherein each decrosslinking catalyst is a compound of formula (I), formula (II), or formula (III), as defined herein; and (ii) instructions for decrosslinking a formaldehyde cross-linked biological sample, instructing a user to simultaneously contact the sample with a first solution comprising the protease and a second solution comprising the decrosslinking catalyst.

### BRIEF DESCRIPTION OF THE DRAWINGS

FIG. 1 shows exemplary cross-links formed when formalin is used to fix tissues, including protein-protein crosslinks and protein-DNA crosslinks.

FIG. 2 shows a current DNA extraction protocol compared to a workflow according to methods described herein.

FIG. 3 shows exemplary workflows used to screen decrosslinking catalyst compounds, along with positive and negative controls.

FIG. 4 shows quantity and quality of DNA yields purified from healthy FFPE colon tissue using decrosslinking catalyst compounds along with positive and negative controls, as determined by the ProNex® DNA QC Assay (75 bp, 150 bp, and 300 bp qPCR amplicons).

FIGS. 5A-5C show quantity and quality of DNA sequencing libraries made with DNA purified in FIG. 4, using Illumina's AmpliSeq for Cancer HotSpot Panel v2. FIG. 5A shows molecular weight of libraries as determined by electrophoresis with Agilent TapeStation. FIG. 5B shows quantification of electrophoresed libraries. FIG. 5C shows quantity of libraries as determined by qPCR with ProNex® NGS Library Quantification Kit.

FIGS. 6A-6B show concordance of single-nucleotide variant counts with DNA purified from FIG. 5, as determined by sequencing with an Illumina MiSeq. FIG. 6A shows total variant allele counts with frequencies above 1%. FIG. 6B shows variant allele frequencies between DNA purified with rapid decrosslinking catalysts, positive control, and sequencing control gDNA.

FIG. 7 shows amplifiable DNA yields from FFPE rapidly decrosslinked with catalyst compounds at 80° C. for 10, 20, and 30 minutes.

FIGS. 8A-8C show determination of optimal pH for rapid decrosslinking reactions with catalyst compounds.

FIG. 9 shows amplifiable DNA yields from FFPE rapidly decrosslinked with 20 mM, 10 mM, and 5 mM of catalyst HCl salt compounds across a range of pH lysis buffers.

FIG. 10 shows screening methods for determining whether decrosslinking catalyst compounds are compatible with proteinase K digestion, including positive and negative controls.

FIG. 11 shows total amplifiable DNA yields (ng) from workflow described in FIG. 7, as determined by a duplex qPCR assay.

FIGS. 12A-12B show proteinase K (ProK) digestion time effects on DNA yield. FIG. 12A shows a schematic of the experimental design. FIG. 12B shows total amplifiable DNA yields (ng), as determined by a duplex qPCR assay and the ProNex® QC qPCR assay (75 bp, 150 bp, and 300 bp amplicons).

FIGS. 13A-13B show FFPE DNA yields from a single-step ProK digestion and decrosslinking incubation. FIG. 13A shows a schematic of the experimental design. FIG. 13B shows total amplifiable DNA yields (ng), as determined by a duplex qPCR assay.

FIGS. 14A-14C show purified DNA yields from a rapid decrosslinking reaction with Cpd 1. DNA was purified from thirteen human FFPE tissues and assessed using an amplification assay (FIG. 14A), a dsDNA dye-binding assay (FIG. 14B), and UV-Vis absorbance (FIG. 14C).

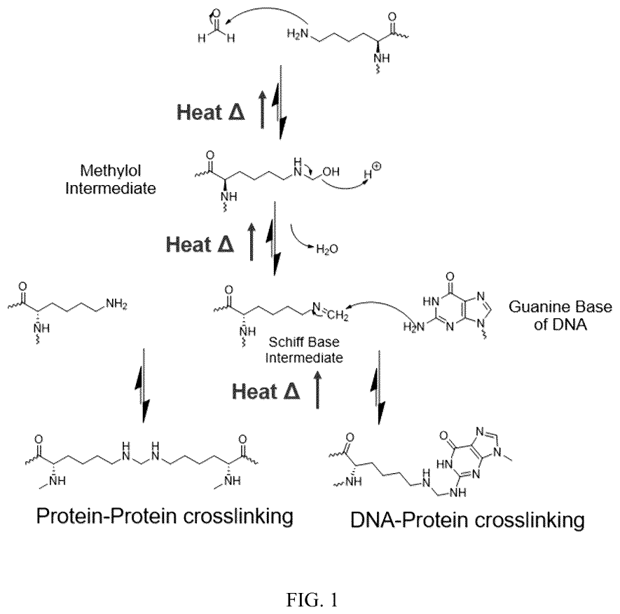

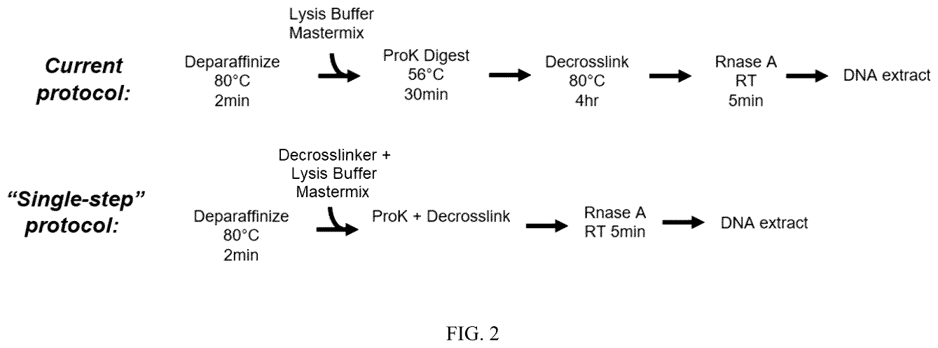

### DETAILED DESCRIPTION

Provided herein are compositions, methods, and kits for decrosslinking formaldehyde cross-linked biological samples, such as FFPE tissue samples. In some embodiments, the samples can be decrosslinked in significantly shorter times than those currently used, as both decrosslinking and protease digestion can be conducted in a single step. For example, in some embodiments, FFPE samples can be efficiently decrosslinked in only 30 minutes, without sacrificing sample quality.

### Definitions

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics, and protein and nucleic acid chemistry and hybridization described herein are those that are well known and commonly used in the art. The meaning and scope of the terms should be clear: in the event, however of any latent ambiguity, definitions provided herein take precedent over any dictionary or extrinsic definition. Further, unless otherwise required by context, singular terms shall include pluralities and plural terms shall include the singular.

As used herein and in the appended claims, the singular forms “a,” “an,” and “the” include plural reference unless the context clearly dictates otherwise.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1. 6.2, 6.3, 6.4, 6.5, 6.6, 6.7, 6.8, 6.9, and 7.0 are explicitly contemplated.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Sorrell, Organic Chemistry, 2^{nd} edition, University Science Books, Sausalito, 2006; Smith, March's Advanced Organic Chemistry: Reactions, Mechanism, and Structure, 7^{th} Edition, John Wiley & Sons, Inc., New York, 2013; Larock, Comprehensive Organic Transformations, 3^{rd} Edition, John Wiley & Sons, Inc., New York, 2018; and Carruthers, Some Modern Methods of Organic Synthesis, 3^{rd} Edition, Cambridge University Press, Cambridge, 1987; the entire contents of each of which are incorporated herein by reference.

As used herein, the term “alkyl” refers to a radical of a straight or branched saturated hydrocarbon chain. The alkyl chain can include, e.g., from 1 to 24 carbon atoms (C₁-C₂₄ alkyl), 1 to 16 carbon atoms (C₁-C₁₆ alkyl), 1 to 14 carbon atoms (C₁-C₁₄ alkyl), 1 to 12 carbon atoms (C₁-C₁₂ alkyl), 1 to 10 carbon atoms (C₁-C₁₀ alkyl), 1 to 8 carbon atoms (C₁-C₈ alkyl), 1 to 6 carbon atoms (C₁-C₆ alkyl), 1 to 4 carbon atoms (C₁-C₄ alkyl), 1 to 3 carbon atoms (C₁-C₃ alkyl), or 1 to 2 carbon atoms (C₁-C₂ alkyl). Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl, n-undecyl, and n-dodecyl.

As used herein, the term “alkenyl” refers to a radical of a straight or branched hydrocarbon chain containing at least one carbon-carbon double bond and no triple bonds. The double bond(s) may be located at any position(s) with the hydrocarbon chain. The alkenyl chain can include, e.g., from 2 to 24 carbon atoms (C₂-C₂₄ alkenyl), 2 to 16 carbon atoms (C₂-C₁₆ alkenyl), 2 to 14 carbon atoms (C₂-C₁₄ alkenyl), 2 to 12 carbon atoms (C₂-C₁₂ alkenyl), 2 to 10 carbon atoms (C₂-C₁₀ alkenyl), 2 to 8 carbon atoms (C₂-Cs alkenyl), 2 to 6 carbon atoms (C₂-C₆ alkenyl), 2 to 4 carbon atoms (C₂-C₄ alkenyl), 2 to 3 carbon atoms (C₂-C₃ alkenyl), or 2 carbon atoms (C₂ alkenyl). Representative examples of alkenyl include, but are not limited to, ethenyl, 1-propenyl, 2-propenyl, 1-butenyl, 2-butenyl, butadienyl, 2-methyl-2-propenyl, 3-butenyl, pentenyl, pentadienyl, hexenyl, heptenyl, octenyl, octatrienyl, and the like.

As used herein, the term “alkynyl” means a radical of a straight or branched hydrocarbon chain containing at least one carbon-carbon triple bond. The alkynyl chain can include, e.g., from 2 to 24 carbon atoms (C₂-C₂₄ alkynyl), 2 to 16 carbon atoms (C₂-C₁₆ alkynyl), 2 to 14 carbon atoms (C₂-C₁₄ alkynyl), 2 to 12 carbon atoms (C₂-C₁₂ alkynyl), 2 to 10 carbon atoms (C₂-C₁₀ alkynyl), 2 to 8 carbon atoms (C₂-C₈ alkynyl), 2 to 6 carbon atoms (C₂-C₆ alkynyl), 2 to 4 carbon atoms (C₂-C₄ alkynyl), 2 to 3 carbon atoms (C₂-C₃ alkynyl), or 2 carbon atoms (C₂ alkynyl). The triple bond(s) may be located at any position(s) with the hydrocarbon chain. Representative examples of alkynyl include, but are not limited to, ethynyl, 1-propynyl, 2-propynyl, 1-butynyl, 2-butynyl, and the like.

As used herein, the term “alkoxy” refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

As used herein, the term “alkylthio” refers to an alkyl group, as defined herein, appended to the parent molecular moiety through a sulfur atom. Representative examples of alkoxy include, but are not limited to, methylthio, ethylthio, and propylthio.

As used herein, the term “amino” refers to a group —NR^{x}R^{y}, wherein R^{x} and R^{y} are selected from hydrogen and alkyl (e.g., C₁-C₄ alkyl). A group —NH(alkyl) may be referred to herein as “alkylamino” and a group —N(alkyl)₂ may be referred to herein as “dialkylamino.”

As used herein, the term “aryl” refers to a radical of a monocyclic, bicyclic, or tricyclic 4n+2 aromatic ring system (e.g., having 6, 10, or 14 π electrons shared in a cyclic array) having 6-14 ring carbon atoms and zero heteroatoms (“C₆-C₁₄ aryl”). In some embodiments, an aryl group has six ring carbon atoms (“C₆ aryl”, i.e., phenyl). In some embodiments, an aryl group has ten ring carbon atoms (“C₁₀ aryl”, e.g., naphthyl such as 1-naphthyl and 2-naphthyl). In some embodiments, an aryl group has fourteen ring carbon atoms (“C₁₄ aryl”, e.g., anthracenyl and phenanthrenyl).

As used herein, the term “carboxy” refers to a —COOH group.

As used herein, the term “carboxyalkyl” refers to an alkyl group, as defined herein, in which at least one hydrogen atom is replaced with a carboxy group. Representative examples of carboxyalkyl include, but are not limited to, carboxymethyl, 2-carboxyethyl, and 3-carboxypropyl.

As used herein, the term “cycloalkyl” refers to a radical of a saturated carbocyclic ring system containing three to ten carbon atoms and zero heteroatoms. The cycloalkyl may be monocyclic, bicyclic, bridged, fused, or spirocyclic. Representative examples of cycloalkyl include, but are not limited to, cyclopropyl, cyclobutyl, cyclopentyl, cyclohexyl, cycloheptyl, cyclooctyl, cyclononyl, cyclodecyl, adamantyl, bicyclo[2.2.1]heptanyl, bicyclo[3.2.1]octanyl, and bicyclo[5.2.0]nonanyl.

As used herein, the term “ester” refers to a group —COOR, wherein R is an alkyl group as defined herein (e.g., a C₁-C₆, C₁-C₄, or C₁-C₃ alkyl group).

As used herein, the term “halogen” or “halo” refers to F, Cl, Br, or I.

As used herein, the term “haloalkyl” refers to an alkyl group, as defined herein, in which at least one hydrogen atom (e.g., one, two, three, four, five, six, seven or eight hydrogen atoms) is replaced with a halogen. In some embodiments, each hydrogen atom of the alkyl group is replaced with a halogen (“perhaloalkyl”). Representative examples of haloalkyl include, but are not limited to, fluoromethyl, difluoromethyl, trifluoromethyl, 2-fluoroethyl, 2,2,2-trifluoroethyl, and 3,3,3-trifluoropropyl.

As used herein, the term “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

As used herein, the term “heterocyclyl” refers to a radical of a 3- to 10-membered non-aromatic ring system having ring carbon atoms and 1 to 4 ring heteroatoms, wherein each heteroatom is independently selected from nitrogen, oxygen, sulfur, selenium, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Exemplary 3-membered heterocyclyl groups containing one heteroatom include, without limitation, azirdinyl, oxiranyl, and thiorenyl. Exemplary 4-membered heterocyclyl groups containing one heteroatom include, without limitation, azetidinyl, oxetanyl, and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin-2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8- membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

As used herein, the term “hydroxy” or “hydroxyl” refers to an —OH group.

As used herein, the term “hydroxyalkyl” refers to an alkyl group, as defined herein, in which at least one hydrogen atom is replaced with a hydroxy group. Representative examples of hydroxyalkyl include, but are not limited to, hydroxymethyl, 2-hydroxyethyl, and 3-hydroxypropyl.

As used herein, the term “oxo” refers to a group ═O.

As used herein, the term “thiol” refers to an —SH group.

When a group or moiety can be substituted, the term “substituted” indicates that one or more (e.g., 1, 2, 3, 4, 5, or 6; in some embodiments 1, 2, or 3; and in other embodiments 1 or 2) hydrogens on the group indicated in the expression using “substituted” can be replaced with a selection of recited indicated groups or with a suitable substituent group known to those of skill in the art (e.g., one or more of the groups recited below), provided that the designated atom's normal valence is not exceeded. Substituent groups include, but are not limited to, alkyl, alkenyl, alkynyl, alkoxy, acyl, amino, amido, amidino, aryl, azido, carbamoyl, carboxy, carboxyalkyl, cyano, cycloalkyl, cycloalkenyl, ester, guanidino, halo, haloalkyl, haloalkoxy, heteroalkyl, heteroaryl, heterocyclyl, hydroxy, hydroxyalkyl, hydrazino, imino, oxo, nitro, phosphate, phosphonate, sulfonic acid, thiol, thione, or combinations thereof.

As used herein, the term “simultaneous” means, in reference to multiple steps, that the steps are conducted at the same time, or that the steps are conducted sequentially without a removal or washing step in between. For example, when a sample is simultaneously contacted with two compounds A and B, the sample is either contacted with compounds A and B at the same time (e.g., with a single composition comprising both compounds A and B), or the sample is first contacted with compound A and subsequently contacted with compound B without removing compound A or washing the sample, or the sample is first contacted with compound B and subsequently contacted with compound A without removing compound B or washing the sample.

As used herein, in chemical structures the indication:

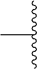

represents a point of attachment of one moiety to another moiety (e.g., a substituent group to the rest of the compound).

When bivalent substituent groups are specified by their conventional chemical formulae, written from left to right, such indication also encompasses substituent groups resulting from writing the structure from right to left. For example, if a bivalent group is shown as —CH₂O—, such indication also encompasses —OCH₂—; similarly, —OC(O)NH— also encompasses —NHC(O)O—.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

### Decrosslinking Catalysts

Disclosed herein are compositions, methods, and kits for decrosslinking formaldehyde cross-linked biological samples, such as FFPE samples. The compositions, methods, and kits, described in further detail below, comprise a decrosslinking catalyst, which is a compound of formula (I), a compound of formula (II), or a compound of formula (III).

In some embodiments, the decrosslinking catalyst is a compound of formula (I):

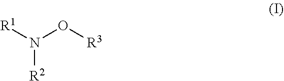

or a salt thereof,
wherein:
R¹ is selected from H and C₁-C₆ alkyl;
R² is selected from H, C₁-C₆ alkyl, aryl, heteroaryl, aryl-C₁-C₄-alkyl, and heteroaryl-C₁-C₄-alkyl;
R³ is selected from H, C₁-C₆ alkyl, and —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or more substituents independently selected from halo, C₁-C₄ alkyl, hydroxy, C₁-C₄ alkoxy, —NR^{a1}R^{b1}, —C(O)NR^{c1}R^{d1}, —COOR^{e1}, and —SO₂NR^{f1}R^{g1};
wherein each alkyl is unsubstituted or substituted with 1 or more substituents independently selected from halo, hydroxy, C₁-C₄ alkoxy, thiol, C₁-C₄ alkylthio, —NR^{a1}R^{b1}, —C(O)NR^{c1}R^{d1}, —COOR^{e1}, —SO₂NR^{f1}R^{g1}, —ONR^{h1}Rⁱ¹, —NR^{j1}OR^{k1}, —PO₃H₂, —SO₃H, optionally substituted C₃-C₆ cycloalkyl, and optionally substituted 3- to 6-membered heterocyclyl; and
wherein R^{a1}, R^{b1}, R^{c1}, R^{d1,} R^{e1}, R^{f1}, R^{g1}, R^{h1}, Rⁱ¹, R^{j1}, and R^{k1} are each independently selected from H, C₁-C₄ alkyl, C₁-C₄ hydroxyalkyl, and C₁-C₄ carboxyalkyl;
wherein R¹ and R², together with the nitrogen atom to which they are attached, are optionally taken together to form an optionally substituted 4- to 8-membered ring; and
wherein R² and R³, together with the atoms to which they are attached, are taken together to form an optionally substituted 4- to 8-membered ring.

In some embodiments: R¹ is H; R² is selected from H, C₁-C₆ alkyl, —CH₂-aryl, and —CH₂-heteroaryl; and R³ is selected from H, C₁-C₆ alkyl, —X—R⁴, wherein X is selected from —C(O)— and —SO₂—, and R⁴ is selected from C₁-C₆-alkyl, aryl, and heteroaryl; wherein each aryl and heteroaryl is independently unsubstituted or substituted with 1 or 2 substituents independently selected from halo, hydroxy and C₁-C₄ alkoxy; and wherein each alkyl is independently unsubstituted or substituted with 1 substituent selected from hydroxy, C₁-C₄ alkoxy, —NR^{a1}R^{b1}, —COOR^{e1}, —ONR^{h1}Rⁱ¹, and —NR^{j1}OR^{k1}, wherein R^{a1}, R^{b1}, R^{e1}, R^{h1}, Rⁱ¹, R^{j1}, and R^{k1} are each independently selected from H and —CH₃.

In some embodiments: R¹ is H; R² is selected from H and C₁-C₆ alkyl; and R³ is selected from H and C₁-C₆ alkyl; wherein each alkyl is independently unsubstituted or substituted with 1 substituent selected from hydroxy, —NR^{a1}R^{b1}, —COOR^{e1}, —ONR^{h1}Rⁱ¹, and —NR^{j1}OR^{k1}, wherein R^{a1}, R^{b1}, R^{e1}, R^{h1}, Rⁱ¹, R^{j1}, and R^{k1} are each independently selected from H and —CH₃. In some embodiments: R¹ is H; R² is selected from H and C₁-C₂ alkyl; and R³ is selected from H and C₁-C₂ alkyl; wherein each alkyl is independently unsubstituted or substituted with 1 substituent selected from hydroxy, —NR^{a1}R^{b1}, —COOR^{e1}, —ONR^{h1}Rⁱ¹, and —NR^{j1}OR^{k1}, wherein R^{a1}, R^{b1}, R^{e1}, R^{h1,} Rⁱ¹, R^{j1}, and R^{k1} are each independently selected from H and —CH₃. In some embodiments: R¹ is H; R² is selected from H, —CH₃, —CH₂CH₃, —CH₂CH₂OH, and —CH₂CH₂NHOH; and R³ is selected from H, —CH₃, —CH₂CH₃, —CH₂CH₂OH, —CH₂CH₂NH₂, —CH₂CH₂ONH₂, —CH₂CH₂ONHCH₃, and —CH₂COOH. In some embodiments, the compound of formula (I) is selected from:

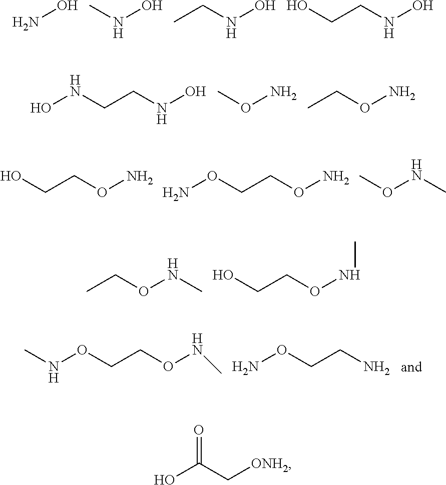

and salts thereof.

In some embodiments: R¹ is H; R² is selected from H and C₁-C₆ alkyl; and R³ is —X—R⁴, wherein X is selected from —C(O)— and —SO₂—, and R⁴ is selected from C₁-C₆-alkyl and aryl; wherein each alkyl is independently unsubstituted or substituted with 1 or 2 substituents independently selected from hydroxy and —COOR^{e1}, wherein R^{e1} is selected from H and —CH₃; and each aryl and heteroaryl is independently unsubstituted or substituted with 1 or 2 substituents independently selected from halo, hydroxy and C₁-C₄ alkoxy. In some embodiments: R¹ is H; R² is selected from H and C₁-C₄ alkyl, wherein the alkyl is unsubstituted or substituted with 1 substituent selected from —OH and —COOH; and R³ is —X—R⁴, wherein X is selected from —C(O)— and —SO₂—, and R⁴ is selected from —CH₃ and phenyl, wherein the phenyl is unsubstituted or substituted with 1 substituent selected from methoxy, hydroxy, and halo. In some embodiments: R¹ is H; R² is selected from H, —CH₃, —CH₂CH₂CH₂CH₃, —CH₂COOH, and —CH₂CH₂OH; and R² is selected from —COCH₃, —COPh, —CO(4-methoxyphenyl), —SO₂CH₃, and —SO₂Ph. In some embodiments, the compound of formula (I) is selected from:

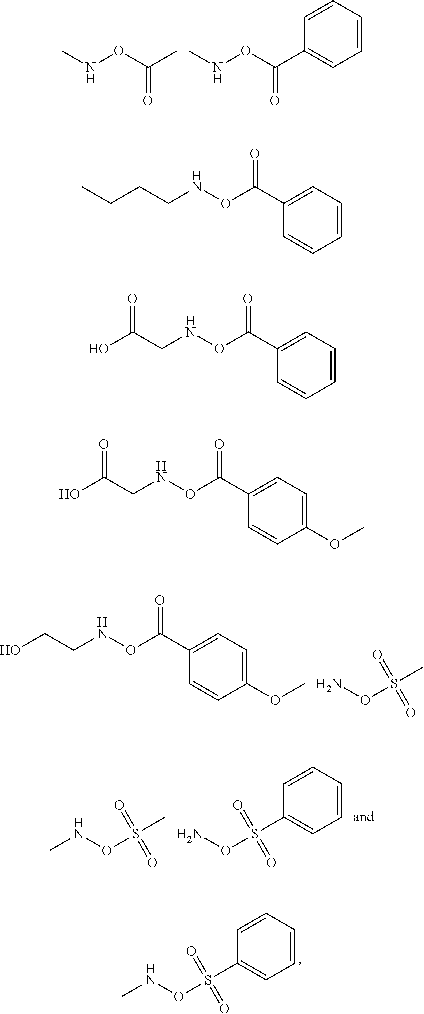

and salts thereof.

In some embodiments: R¹ is H; R² is selected from aryl-C₁-C₄-alkyl and heteroaryl-C₁-C₄-alkyl, each of which is independently unsubstituted or substituted with 1 or 2 substituents independently selected from halo, hydroxy, and C₁-C₄ alkoxy; and R³ is H. In some embodiments: R¹ is H; R² is selected from —CH₂-aryl and —CH₂-heteroaryl, each of which is independently unsubstituted or substituted with 1 or 2 substituents independently selected from halo, hydroxy and C₁-C₄ alkoxy; and R³ is H. In some embodiments: R¹ is H; R²-CH₂-phenyl and —CH₂-heteroaryl, wherein the heteroaryl is a monocyclic heteroaryl having one heteroatom selected from N, O, and S (e.g., pyridyl, furanyl, or thiophenyl), and wherein the phenyl or the heteroaryl is unsubstituted or substituted with 1 or 2 substituents independently selected from chloro, hydroxy, and methoxy; and R³ is H. In some embodiments, the compound of formula (I) is selected from:

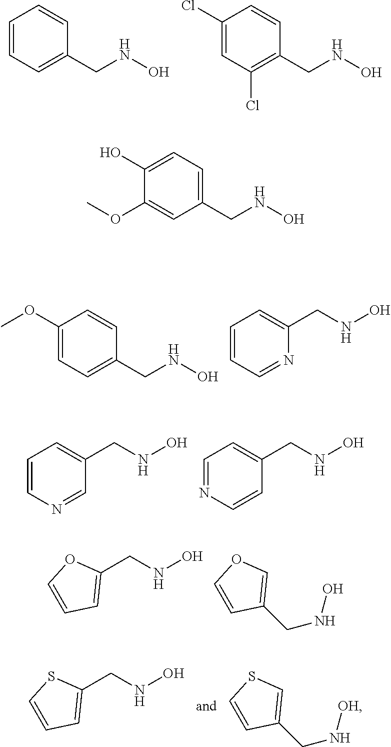

and salts thereof.

In some embodiments, R¹ and R², together with the nitrogen atom to which they are attached, are optionally taken together to form an optionally substituted 4- to 8-membered ring; and R³ is H. In some embodiments, R¹ and R², together with the nitrogen atom to which they are attached, are taken together form a saturated 4- to 8-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and R³ is H. In some embodiments, R¹ and R², together with the nitrogen atom to which they are attached, are taken together form a saturated 4- to 6-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo; and R³ is H. In some embodiments, R¹ and R², together with the nitrogen atom to which they are attached, are taken together form a saturated 5- to 6-membered ring that is unsubstituted or substituted with 1 or 2 substituents independently selected from hydroxy, —CH₂OH, carboxy, —CH₂COOH, and oxo; and R³ is H. In some embodiments, R¹ and R², together with the nitrogen atom to which they are attached, are taken together form a pyrrolidine, piperidine, or morpholine ring, each of which is unsubstituted or substituted with 1 or 2 substituents independently selected from hydroxy, —CH₂OH, carboxy, —CH₂COOH, and oxo; and R³ is H. In some embodiments, the compound of formula (I) is selected from:

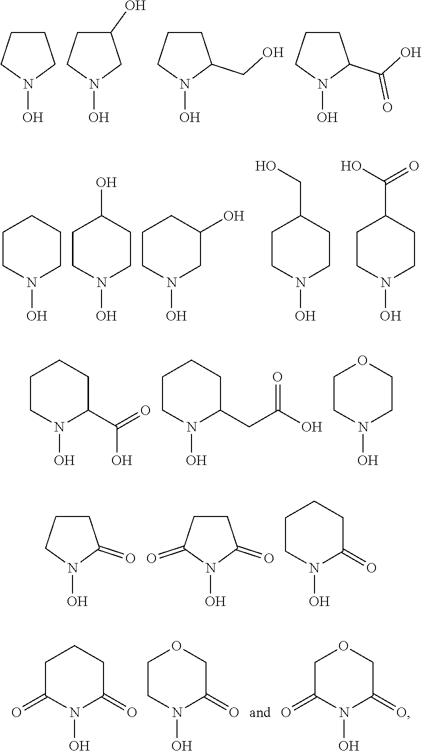

and salts thereof.

In some embodiments, R¹ is H, and R² and R³, together with the atoms to which they are attached, are taken together to form an optionally substituted 4- to 8-membered ring. In some embodiments, R¹ is H, and R² and R³, together with the atoms to which they are attached, are taken together to form a saturated 4- to 8-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R¹ is H, and R² and R³, together with the atoms to which they are attached, are taken together to form a saturated 4- to 7-membered ring that is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R¹ is H, and R² and R³, together with the atoms to which they are attached, are taken together to form a saturated 4- to 7-membered ring that is unsubstituted or substituted with 1 or 2 substituents independently selected from hydroxy, —CH₂OH, —CH₂CH₂OH, methoxy, carboxy, and oxo. In some embodiments, R¹ is H, and R² and R³, together with the atoms to which they are attached, are taken together to form a 1,2-oxazetidine, isoxazolidine, 1,2-oxazinane, or 1,2-oxazepane ring, each of which is unsubstituted or substituted with 1 or 2 substituents independently selected from hydroxy, hydroxy-C₁-C₄ alkyl, C₁-C₄-alkoxy, carboxy, carboxy-C₁-C₄ alkyl, and oxo. In some embodiments, R¹ is H, and R² and R³, together with the atoms to which they are attached, are taken together to form a 1,2-oxazetidine, isoxazolidine, 1,2-oxazinane, or 1,2-oxazepane ring, each of which is unsubstituted or substituted with 1 substituent selected from hydroxy, —CH₂OH, —CH₂CH₂OH, methoxy, carboxy, and oxo. In some embodiments, the compound of formula (I) is selected from:

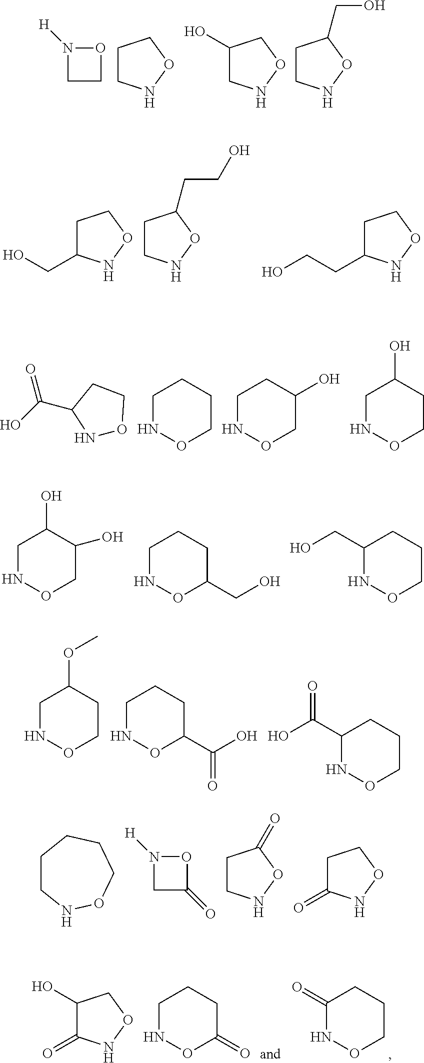

and salts thereof.

In some embodiments, the compound of formula (I) is selected from:

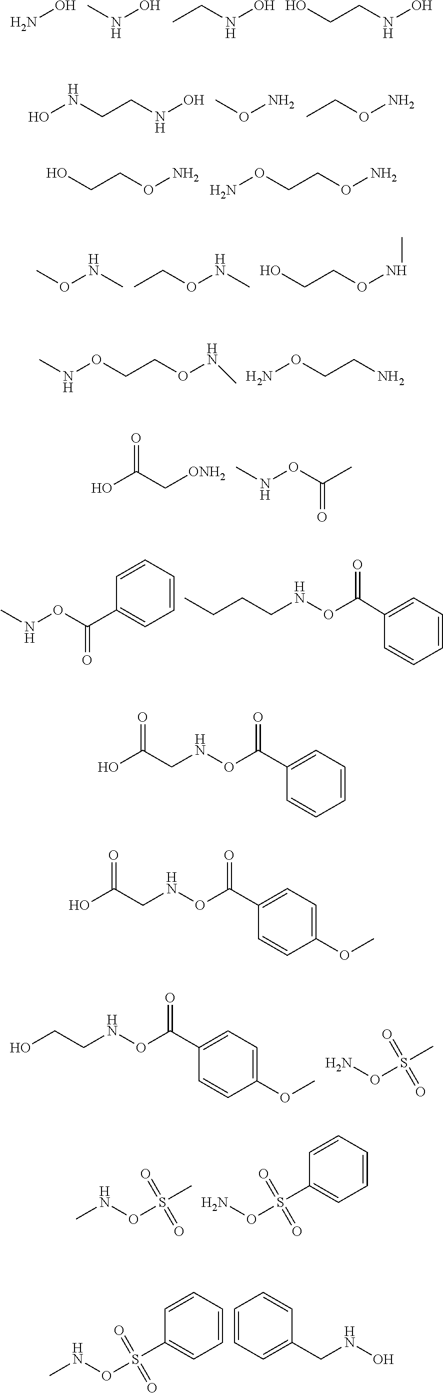

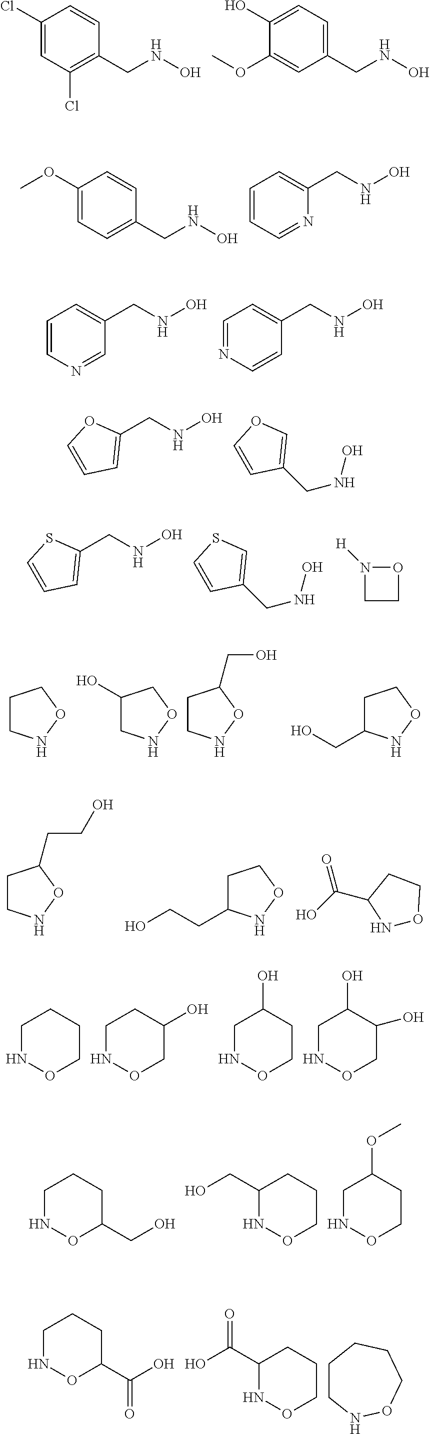

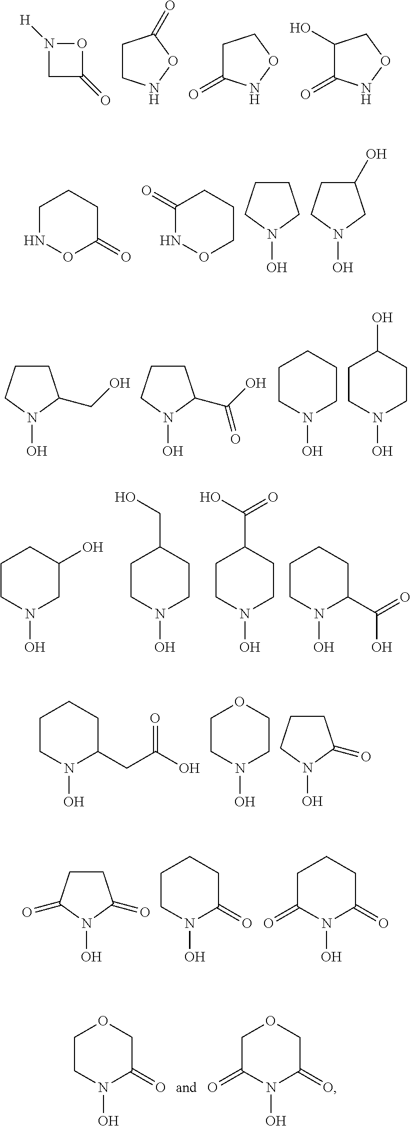

and salts thereof.

In some embodiments, the decrosslinking catalyst is a compound of formula (II):

A—NR^{x}R^{y} (II)
or a salt thereof, wherein:
A is selected from H, C₁-C₆-alkyl, aryl, heteroaryl, C₃-C₆-cycloalkyl, aryl-C₁-C₄-alkyl, heteroaryl-C₁-C₄-alkyl, and —Q—R⁵;
Q is selected from —NR^{a2}—, —NR^{b2}CO—, —SO₂—, —SO₂NR^{c2}—, and —NR^{d2}COCONR^{e2}NR^{f2}—;
R⁵ is selected from H, C₁-C₆-alkyl, aryl, heteroaryl, and C₃-C₆-cycloalkyl;
R^{a2}, R^{b2}, R^{c2}, R^{d2}, R^{e2}, and R^{f2} are each independently selected from H and C₁-C₆ alkyl;
R^{x} and R^{y} are each independently selected from H and C₁-C₆ alkyl; or R^{x} and R^{y}, together with the nitrogen atom to which they are attached, form an optionally substituted 4- to 8-membered ring;
wherein each aryl or heteroaryl is unsubstituted or substituted independently selected from halo, hydroxyl, C₁-C₄ alkyl, C₁-C₄ alkoxy, C₁-C₄ hydroxyalkyl, —P(O)(OH)₂, —B(OH)₂, —COOR^{g2}, —CONR^{h2}Rⁱ², —SO₂NR^{j2}R^{k2}, and —SO₂OR^{l2}; and
wherein each alkyl or cycloalkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOR^{g2}, —PO₃H₂, —SO₂OR^{l2}, and —NR^{m2}Rⁿ²;
wherein each R^{g2}, R^{h2}, Rⁱ², R^{j2}, R^{k2}, R^{l2}, R^{m2}, and Rⁿ² is independently selected from H and methyl.

In some embodiments, R^{x} is H, and R^{y} is selected from H and —CH₃. In some embodiments, R^{x} and R^{y} are each H.

In some embodiments, A is selected from phenyl, monocyclic heteroaryl, and C₅-C₆-cycloalkyl, each of which is unsubstituted or substituted with 1 or 2 substituents independently selected from C₁-C₄ alkyl, C₁-C₄ alkoxy, —P(O)(OH)₂, —B(OH)₂, and —COOH. In some embodiments, A is selected from phenyl and cyclohexyl, each of which is unsubstituted or substituted with 1 or 2 substituents independently selected from methyl, methoxy, —P(O)(OH)₂, —B(OH)₂, and —COOH. In some embodiments, the compound of formula (II) is selected from:

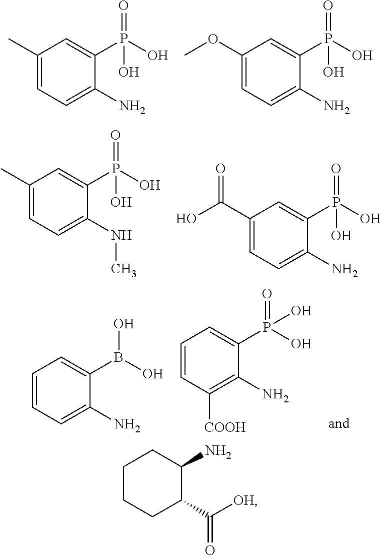

and salts thereof.

In some embodiments: A is selected from C₁-C₆-alkyl and —Q—R⁵; Q is selected from —NR^{a2}—, —NR^{b2}CO—, —SO₂—, and —NR^{d2}COCONR^{e2}NR^{f2}; and R⁵ is H, C₁-C₆-alkyl, phenyl, or a monocyclic heteroaryl; wherein each alkyl is unsubstituted or substituted with 1 or 2 substituents independently selected from hydroxy, —P(O)(OH)₂, —COOR^{g2}, and —NR^{m2}Rⁿ²; and wherein R^{a2}, R^{b2}, R^{d2}, R^{e2}, R^{f2}, R^{g2}, R^{m2}, and Rⁿ² are each H. In some embodiments, A is selected from —CH₂CH₂P(O)(OH)₂, —CH₂CH(OH)CH₂OH, —NHCH₃, —NHC(O)CH₃, —NHC(O)CH₂OH, —NHC(O)CH₂CH₂CH(COOH)NH₂, —NC(O)C(O)NHNH₂, NHC(O)-pyridyl, and —SO₂-phenyl. In some embodiments, the compound of formula (II) is selected from:

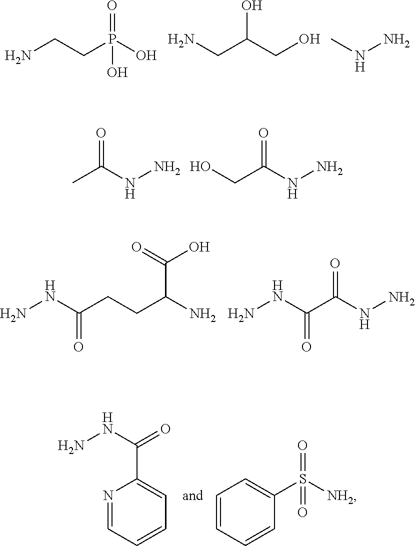

and salts thereof.

In some embodiments, R^{x} and R^{y}, together with the nitrogen atom to which they are attached, form an optionally substituted 4- to 8-membered ring. In some embodiments, R^{x} and R^{y}, together with the nitrogen atom to which they are attached, form a 6-membered ring selected from morpholine, thiomorpholine, selenomorpholine, and piperazine, each of which is unsubstituted or substituted with 1 or 2 oxo groups; A is selected from H, —CH₃, —CH₂CH₃, —CH₂COOH, —CH₂CH₂OH, and —Q—R⁵; Q is—NR^{a2}— or —NR^{b2}CO—, wherein R^{a2} and R^{b2} are each H; and R⁵ is H or —CH₃. In some embodiments, the compound of formula (II) is selected from:

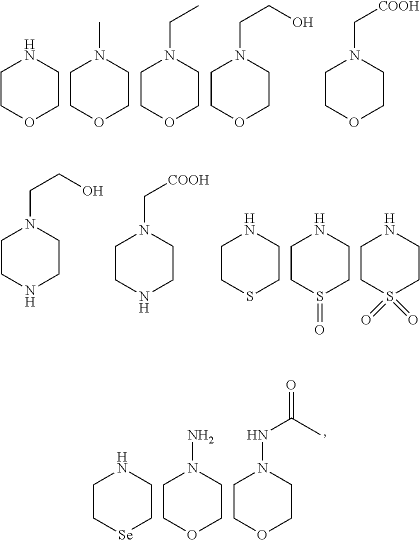

and salts thereof.

In some embodiments, the compound of formula (II) is selected from:

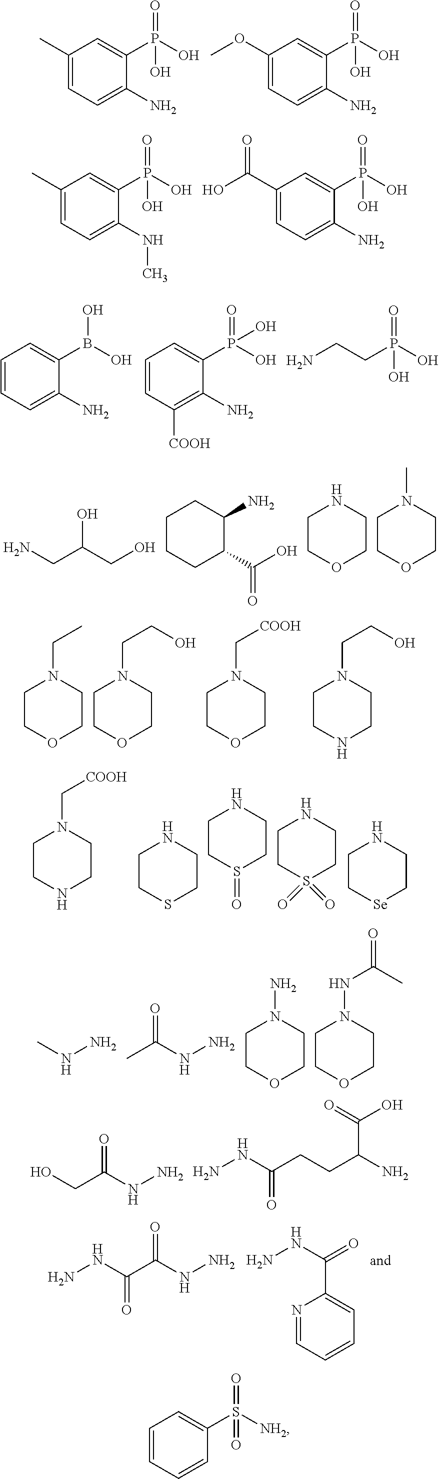

and salts thereof.

In some embodiments, the decrosslinking catalyst is a compound of formula (III):

B—X (III)
or a salt thereof, wherein:
B is aryl, heteroaryl, C₁-C₆ alkyl, aryl-C₁-C₄-alkyl-, aryl-C₂-C₄-alkoxy-, heteroaryl-C₁-C₄-alkyl-, and heteroaryl-C₂-C₄-alkoxy-; and
X is selected from —COOH, —PO₃H₂, —B(OH)₂, and —SO₃H;
wherein each aryl and heteroaryl is independently unsubstituted or substituted with 1 or more substituents independently selected from halo, C₁-C₄ alkyl, C₁-C₄ alkoxy, hydroxy, C₁-C₄ hydroxyalkyl, —NR^{a3}R^{b3}, —COOR^{c3}, —SO₃R^{d3}, —PO₃H₂, and —B(OH)₂;
wherein each alkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOH, —PO₃H₂ and —SO₃H; and
wherein R^{a3}, R^{b3}, R^{c3}, and R^{d3} are each independently selected from H, —CH₃, and —CH₂CH₃.

In some embodiments: B is aryl or heteroaryl, each of which is unsubstituted or substituted with 1 or 2 substituents independently selected from halo, methyl, methoxy, —COOH, and —PO₃H₂; and X is selected from —COOH, —PO₃H₂, and —SO₃H. In some embodiments: B is phenyl or a monocyclic heteroaryl having one heteroatom selected from N, O, and S; each of which is unsubstituted or substituted with 1 substituent selected from methyl, methoxy, fluoro, chloro, —COOH, and —PO₃H₂; and X is selected from —COOH, —PO₃H₂, and —SO₃H. In some embodiments, B is selected from phenyl, pyridyl, furanyl, and thiophenyl, each of which is unsubstituted or substituted with 1 substituent selected from methyl, methoxy, fluoro, chloro, —COOH, and —PO₃H₂; and X is selected from —COOH, —PO₃H₂, and —SO₃H. In some embodiments, the compound of formula (III) is selected from:

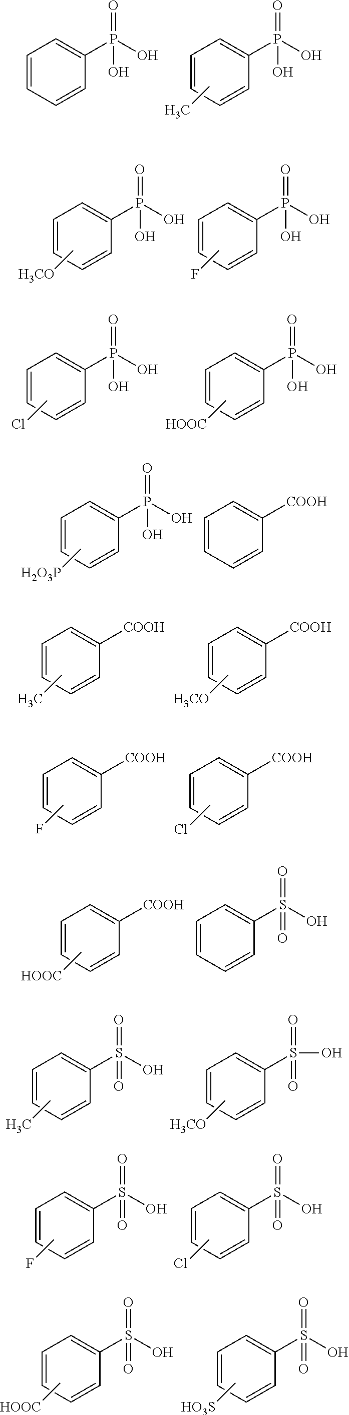

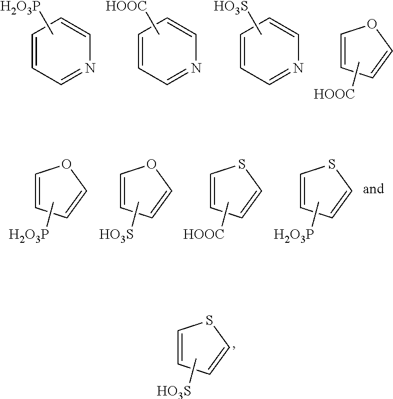

and salts thereof.

In some embodiments, the decrosslinking catalyst is selected from:

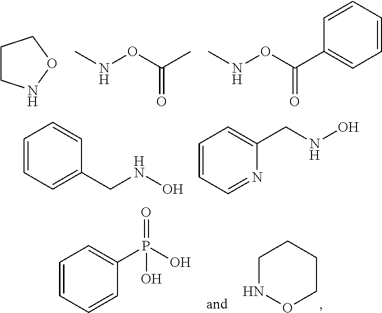

and salts thereof.

In some embodiments, the decrosslinking catalyst is selected from:

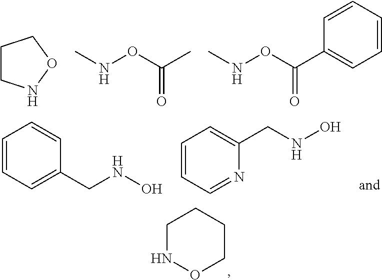

and salts thereof.

In some embodiments, the decrosslinking catalyst (e.g., the compound of formula (I), (II), or (III)) is in the form of a salt. In some embodiments, the decrosslinking catalyst is in the form of an acid addition salt. An acid addition salt can be formed with an inorganic acid or an organic acid. Inorganic acids from which salts can be derived include, for example, hydrochloric acid, hydrobromic acid, sulfuric acid, nitric acid, phosphoric acid, and the like. Organic acids from which salts can be derived include, for example, acetic acid, trifluoroacetic acid, propionic acid, glycolic acid, oxalic acid, maleic acid, malonic acid, succinic acid, fumaric acid, tartaric acid, citric acid, benzoic acid, mandelic acid, methanesulfonic acid, ethanesulfonic acid, toluenesulfonic acid, sulfosalicylic acid, and the like. In some embodiments, the decrosslinking catalyst is in the form of a hydrochloric acid salt.

Unless otherwise stated, structures depicted herein are also meant to include geometric (or conformational) forms of the structure: for example, the R and S configurations for each asymmetric center, Z and E double bond isomers, and Z and E conformational isomers. Therefore, single stereochemical isomers as well as enantiomeric, diastereomeric, and geometric (or conformational) mixtures of the disclosed compounds are within the scope of the disclosure. Unless otherwise stated, all tautomeric forms of the compounds described herein are within the scope of the disclosure.

Additionally, unless otherwise stated, structures depicted herein are also meant to include compounds that differ only in the presence of one or more isotopically enriched atoms. For example, compounds having the disclosed structures including the replacement of hydrogen by deuterium or tritium, or the replacement of a carbon by a ¹³C or ¹⁴C enriched carbon, are within the scope of this disclosure.

### Methods and Compositions

The decrosslinking catalysts disclosed herein (e.g., compounds of formula (I), (II), and (III)) are useful for decrosslinking a formaldehyde cross-linked biological sample in a one-step process, in which the sample is simultaneously contacted with the decrosslinking catalyst and a protease. Accordingly, disclosed herein is method of decrosslinking a formaldehyde cross-linked biological sample, comprising a step of simultaneously contacting the sample with a protease and a decrosslinking catalyst, wherein the decrosslinking catalyst is a compound of formula (I), formula (II), or formula (III) (described in detail above).

In some embodiments, the sample is a formalin-fixed paraffin embedded tissue sample. In some embodiments in which the sample is an FFPE sample, the methods further comprise a step of deparaffinizing the sample prior contacting the sample with the decrosslinking catalyst. Deparaffinizing can be conducted according to standard methods. A common deparaffinization protocol includes incubating the sample with mineral oil at about 80° C. for 1-3 minutes, and optionally repeating the mineral oil treatment one or more additional times to complete the paraffin removal process. Other methods of deparaffinizing samples may also be used, such as treatment with xylenes. In some embodiments, the FFPE sample is not deparaffinized prior to any further treatment steps.

The decrosslinking catalyst aids in removal of formaldehyde cross-links from the sample, to release nucleic acids from the sample. The protease treatment can eliminate contaminating proteins, such as DNases and RNases. In some embodiments, the protease is selected from proteinase K, trypsin, LysC, ProAlanase, and pepsin. In some embodiments, the protease is proteinase K. The step of contacting the sample with the decrosslinking catalyst and the protease can be conducted for a period of time sufficient to decrosslink the sample and cleave proteins in the sample. In some embodiments, the contacting step is conducted for about 5 minutes to about 120 minutes, or about 20 minutes to about 40 minutes. In some embodiments, the contacting step is conducted for about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 70 minutes, about 80 minutes, about 90 minutes, about 100 minutes, about 110 minutes, or about 120 minutes.

The step of contacting the sample with the decrosslinking catalyst and the protease can be conducted at a temperature that is suitable for decrosslinking the sample and cleaving proteins in the sample. In some embodiments, the contacting step is conducted at a temperature of about 20° C. to about 100° C., or about 50° C. to about 80° C. In some embodiments, the contacting step is conducted at ambient (i.e., room) temperature. In some embodiments, the contacting step is conducted at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C.

The step of contacting the sample with the decrosslinking catalyst and the protease can be conducted by contacting the sample with a single solution comprising both the decrosslinking catalyst and the protease, e.g., an aqueous solution comprising the decrosslinking catalyst and the protease. In other embodiments, the step of contacting the sample with the decrosslinking catalyst and the protease can be conducted by contacting the sample with a first solution comprising the protease (e.g., a first aqueous solution comprising the protease) and a second solution comprising the decrosslinking catalyst (e.g., a second aqueous solution comprising the decrosslinking catalyst). The sample can be contacted with the first solution and the second solution at the same time or sequentially, provided that a washing step is not conducted between the sequential contacting steps. In some embodiments, the sample is contacted with the first solution and the second solution within about 30 seconds, within about 1 minute, within about 2 minutes, within about 3 minutes, within about 4 minutes, within about 5 minutes, within about 6 minutes, within about 7 minutes, within about 8 minutes, within about 9 minutes, within about 10 minutes, within about 11 minutes, within about 12 minutes, within about 13 minutes, within about 14 minutes, within about 15 minutes, within about 16 minutes, within about 17 minutes, within about 18 minutes, within about 19 minutes, within about 20 minutes, within about 21 minutes, within about 22 minutes, within about 23 minutes, within about 24 minutes, within about 25 minutes, within about 26 minutes, within about 27 minutes, within about 28 minutes, within about 29 minutes, or within about 30 minutes of each other, in either order, without a washing step in between. In some embodiments, the sample is contacted with the first solution comprising the protease, and subsequently contacted with the second solution comprising the decrosslinking catalyst, with about 30 seconds to about 30 minutes between the contacting steps, with no washing step in between.

The pH of the solution (e.g., the aqueous solution comprising the decrosslinking catalyst and the protease, the first solution, and/or the second solution) after contacting the sample with the solution can be about 4 to about 8.5, or about 4.5 to about 6.5, or about 4.5 to about 6.0. In some embodiments, the pH of the resulting solution after the contacting step (i.e., a step of contacting a cross-linked biological sample with the solution) is about 4.0, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5.

In some embodiments, the aqueous solution or the first solution comprises the decrosslinking catalyst at a concentration of about 1 mM to about 100 mM, or about 5 mM to about 50 mM. In some embodiments, the aqueous solution comprises the decrosslinking catalyst at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM,, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, or about 100 mM. In embodiments in which more than one decrosslinking catalyst is used, each one can be individually present at any of the indicated concentrations.

In some embodiments, the method comprises simultaneously contacting the sample with a protease and more than one decrosslinking catalyst described herein, e.g., two different decrosslinking catalysts, such as two different compounds of formula (I), formula (II), or formula (III), or salts thereof. In some embodiments, the method comprises contacting the sample with two different decrosslinking catalysts, wherein each is a different compound of formula (I), or a salt thereof. In some embodiments, the method comprises contacting the sample with two different decrosslinking catalysts, wherein each is a different compound of formula (II), or a salt thereof. In some embodiments, the method comprises contacting the sample with two different decrosslinking catalysts, wherein each is a different compound of formula (III), or a salt thereof. In some embodiments, the method comprises contacting the sample with two different decrosslinking catalysts, wherein one is a compound formula (I), or a salt thereof, and the other is a compound of formula (II), or a salt thereof. In some embodiments, the method comprises contacting the sample with two different decrosslinking catalysts, wherein one is a compound formula (I), or a salt thereof, and the other is a compound of formula (III), or a salt thereof. In some embodiments, the method comprises contacting the sample with two different decrosslinking catalysts, wherein one is a compound formula (II), or a salt thereof, and the other is a compound of formula (III), or a salt thereof. In some embodiments, the solution (e.g., the aqueous solution comprising the decrosslinking catalyst and the protease, the first solution, and/or the second solution) further comprises a buffer. For example, in some embodiments, the buffer is selected from tris(hydroxymethyl)aminomethane (Tris), 2-(N-morpholino)ethanesulfonic acid (MES), 4-(2-hydroxyethyl)-1-piperazineethanesulfonic acid (HEPES), phosphate-buffered saline, glycine, and citrate. Without wishing to be limited by theory, certain buffers may aid in sequestering/quenching formaldehyde from the decrosslinked FFPE in the sample.

The methods can efficiently decrosslink cross-linked biological samples and degrade proteins within the samples, such as FFPE tissue samples, without sacrificing quality of the nucleic acids contained within the samples. In some embodiments, the methods further comprise a step of extracting one or more components from the sample after the contacting step. In some embodiments, the one or more components are nucleic acids. In some embodiments, the nucleic acids are DNA. In some embodiments, the nucleic acids are RNA. In some embodiments, the one or more components are proteins.

The methods may further include a method of detecting one or more components following its extraction from the sample. For example, when a nucleic acid is extracted from the sample, the nucleic acids can be detected by, for example, nucleotide sequencing or sequence-specific hybridization. In some embodiments, the methods further comprise a step of amplifying one or more nucleic acids from the sample. Any time of amplification may be used, including polymerase chain reaction (PCR), quantitative PCR, real time PCR, hot start PCR, single cell PCR, nested PCR, in situ colony PCR, digital PCR (dPCR), Droplet Digital™ PCR (ddPCR), emulsion PCR, ligase chain reaction (LCR), transcription based amplification system (TAS), nucleic acid sequence-based amplification (NASBA), strand displacement amplification (SDA), rolling circle amplification (RCA), and hyper-branched RCA (HRCA). In some embodiments, the nucleic acid amplification reaction is a multiplex nucleic acid amplification reaction. In some embodiments, the sequencing analysis comprises fragment analysis and/or Sanger sequencing analysis, or Next Generation Sequencing (NGS) analysis.

Other characterization methods include dye binding, absorbance, and enzymatic digestion. For example, dye binding assays use fluorescent dyes specific to nucleic acids and a fluorometer for quantification. Absorbance assays are spectrophotometric assays that rely on nucleic acid's natural light absorbing properties to determine concentration and purity. Enzymatic digestions include applications in which FFPE nucleic acids are digested with enzymes to improve quality or as an intermediate step for further experimentation. For example, Uracil-DNA Glycosylase can improve FFPE DNA quality by excising uracil nucleobases that have originated from cytosine deamination, while S1 Nuclease digests single-stranded DNA and can improve FFPE DNA quality by increasing proportion of double-stranded DNA and reducing artifacts that originate from single-stranded DNA templates.

Also disclosed herein are compositions comprising a protease and a decrosslinking catalyst (e.g., a decrosslinking catalyst of formula (I), (II), or (III), or a salt of any thereof). In some embodiments, the protease is selected from proteinase K, trypsin, LysC, ProAlanase, and pepsin. In some embodiments, the protease is proteinase K.

In some embodiments, the composition is a solution comprising the protease and the decrosslinking catalyst. In some embodiments, the solution has a pH of about 4 to about 8.5 upon adding the solution to a cross-linked biological sample. In some embodiments, the solution has a pH of about 4.5 to about 6.5, or about 4.5 to about 6.0, upon adding the solution to a cross-linked biological sample. In some embodiments, solution has a pH of about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, or about 8.5, upon adding the solution to a cross-linked biological sample.

In some embodiments, the solution comprises the decrosslinking catalyst at a concentration of about 1 mM to about 100 mM, or about 5 mM to about 50 mM. In some embodiments, the solution comprises the decrosslinking catalyst at a concentration of about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM, about 30 mM, about 31 mM, about 32 mM, about 33 mM, about 34 mM, about 35 mM, about 36 mM, about 37 mM, about 38 mM, about 39 mM, about 40 mM, about 41 mM, about 42 mM, about 43 mM, about 44 mM, about 45 mM, about 46 mM, about 47 mM, about 48 mM, about 49 mM, about 50 mM, about 51 mM, about 52 mM, about 53 mM, about 54 mM, about 55 mM, about 56 mM, about 57 mM, about 58 mM, about 59 mM, about 60 mM, about 61 mM, about 62 mM, about 63 mM, about 64 mM, about 65 mM, about 66 mM, about 67 mM, about 68 mM, about 69 mM, about 70 mM, about 71 mM, about 72 mM, about 73 mM, about 74 mM, about 75 mM, about 76 mM, about 77 mM, about 78 mM, about 79 mM, about 80 mM, about 81 mM, about 82 mM, about 83 mM, about 84 mM, about 85 mM, about 86 mM, about 87 mM, about 88 mM, about 89 mM, about 90 mM, about 91 mM, about 92 mM, about 93 mM, about 94 mM, about 95 mM, about 96 mM, about 97 mM, about 98 mM, about 99 mM, or about 100 mM.

In some embodiments, the composition comprises two different decrosslinking catalysts, such as two different compounds of formula (I), formula (II), or formula (III), or salts thereof. In some embodiments, the composition comprises two different decrosslinking catalysts, wherein each is a different compound of formula (I), or a salt thereof. In some embodiments, the composition comprises two different decrosslinking catalysts, wherein each is a different compound of formula (II), or a salt thereof. In some embodiments, the composition comprises two different decrosslinking catalysts, wherein each is a different compound of formula (III), or a salt thereof. In some embodiments, the composition comprises two different decrosslinking catalysts, wherein one is a compound formula (I), or a salt thereof, and the other is a compound of formula (II), or a salt thereof. In some embodiments, the composition comprises two different decrosslinking catalysts, wherein one is a compound formula (I), or a salt thereof, and the other is a compound of formula (III), or a salt thereof. In some embodiments, the composition comprises two different decrosslinking catalysts, wherein one is a compound formula (II), or a salt thereof, and the other is a compound of formula (III), or a salt thereof. In embodiments in which the composition comprises more than one decrosslinking catalyst, each one can be individually present at any of the concentrations indicated above.

In some embodiments, the composition further comprises a formaldehyde cross-linked biological sample, e.g., a formalin-fixed paraffin embedded tissue sample.

Systems and Kits Also disclosed herein are systems and kits for decrosslinking a formaldehyde cross-linked biological sample. The systems and kits comprise a decrosslinking catalyst, such as any compound of formula (I), (II), or (III) described herein (or salts thereof), including those specifically exemplified above, and instructions for decrosslinking a formaldehyde cross-linked biological sample, instructing a user to simultaneously contact the sample with the decrosslinking catalyst and a protease. In some embodiments, the system or kit comprises one or more decrosslinking catalyst disclosed herein (e.g., two different compounds of formula (I), two different compounds of formula (II), two different compounds of formula (III), a compound of formula (I) and a compound of formula (II), a compound of formula (I) and a compound of formula (III), or a compound of formula (II) and a compound of formula (III), or salts of any thereof.

In some embodiments, the system or kit includes the compound either alone or in a solvent such as water, DMSO, or a buffer. When the compound is provided in the absence of a solvent, the system or kit may further include the solvent in which the compound can be dissolved. The system or kit may further comprise one or more reagents used to carry out a decrosslinking reaction and/or a downstream assay such as a detection method described herein.

In some embodiments, the system or kit further comprises other compounds that are used in the methods disclosed herein. For example, in some embodiments, the system or kit further comprises a protease, such as proteinase K. In some embodiments, the system or kit further comprises a compound for conducting a deparaffinization step, such as mineral oil or xylenes.

### EXAMPLES

### Example 1

### Decrosslinking Compound Screening

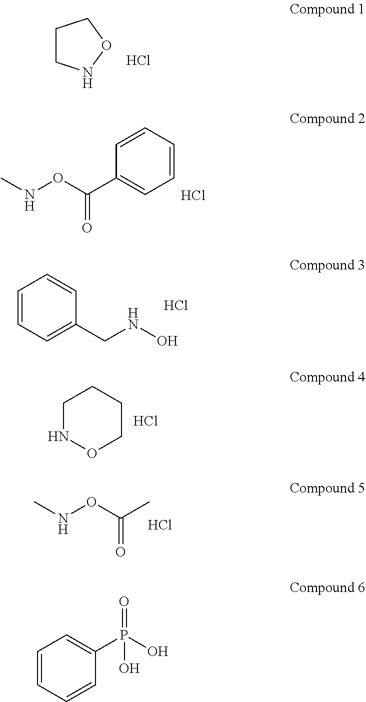

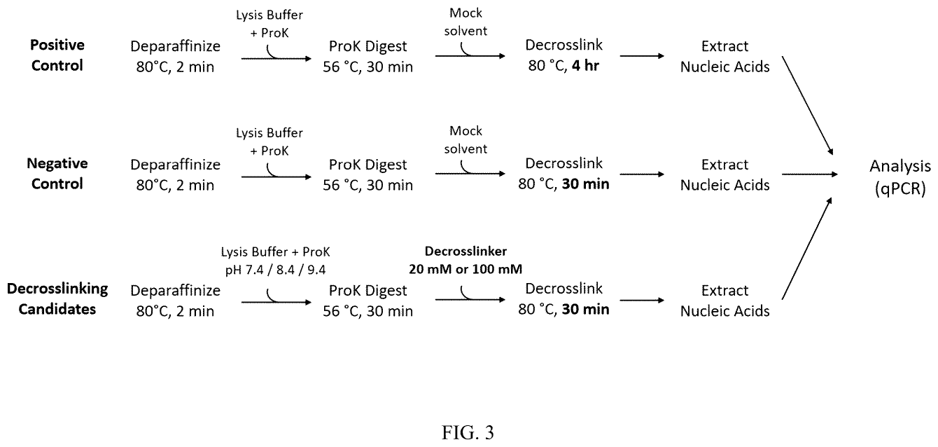

Candidate decrosslinking catalyst compounds, Compounds 1-5, were screened according to the general workflow shown in FIG. 3. (Compound 6 was tested separately.) Compounds were added to a FFPE tissue lysate after a Proteinase K (ProK) digestion (56° C. for 30 minutes), followed by decrosslinking at 80° C. For a positive control, decrosslinking was conducted at 80° C. for 4 hours, and as a negative control, decrosslinking was conducted at 80° C. for 30 minutes. Candidate decrosslinking compounds, at a concentration of either 20 mM or 100 mM were placed into lysates buffered at a pH or 7.4, 8.4, or 9.6, and were incubated with the samples at 80° C. for 30 minutes. Decrosslinked nucleic acids were purified using the Maxwell® RSC DNA FFPE Kit (Promega Cat. No. AS1450) on the Maxwell® RSC 48 Instrument (Promega Cat. No. AS8500) according to the manufacturer's protocol. After extraction of nucleic acids, DNA recovery was determined by qPCR analysis using primers specific to RNaseP (“R”) and telomerase reverse transcriptase (“T”), resulting in 102 bp and 164 bp amplicons, respectively. Screens were conducted with cell pellet artificial FFPE derived from different tissue types and immortalized cell lines. Results are shown in Tables 1 and 2, each of which shows DNA yield (ng) (top) and relative DNA yields normalized against the negative control (bottom). As can be seen, decrosslinking compounds disclosed herein show similar recovery yield at 30 minutes compared with incubation without the decrosslinking compounds at 4 hours.

TABLE 1

| | Decrosslink | Lysis | Test cpd. | Hydroxylamine | | Compound 1 | | Compound 2 | |
|---|---|---|---|---|---|---|---|---|---|
| | Time (hr) | buffer pH | conc. (mM) | R | T | R | T | R | T |
| Pos. Control | 4 | 7.4 | 0 | 383.4 | 76.2 | 755.7 | 1099.6 | 346.9 | 1242.4 |
| Neg. Control | 0.5 | 7.4 | 0 | 15.4 | 2.5 | 50.9 | 20.7 | 121.6 | 161.2 |
| | 0.5 | 7.4 | 20 | 24.6 | 5.1 | 597.3 | 921.4 | 491.9 | 1186.1 |
| | 0.5 | 8.4 | 20 | 23.5 | 5.5 | 514.1 | 1443.9 | 90.8 | 63.1 |
| | 0.5 | 9.6 | 20 | 21.9 | 8.7 | 89.2 | 37.2 | 0.1 | 0.0 |
| | 0.5 | 7.4 | 100 | 122.9 | 54.7 | 14.8 | 0.1 | 0.0 | 0.0 |
| | 0.5 | 8.4 | 100 | 73.1 | 8.4 | 79.9 | 13.5 | 0.0 | 0.0 |
| | 0.5 | 9.6 | 100 | 36.2 | 8.1 | 215.5 | 180.8 | 0.0 | 0.0 |
| Pos. Control | 4 | 7.4 | 0 | 25.0 | 30.0 | 14.8 | 53.1 | 7.0 | 7.7 |
| Neg. Control | 0.5 | 7.4 | 0 | 1.0 | 1.0 | 1.0 | 1.0 | 1.0 | 1.0 |
| | 0.5 | 7.4 | 20 | 1.6 | 2.0 | 11.7 | 44.5 | 4.0 | 7.4 |
| | 0.5 | 8.4 | 20 | 1.5 | 2.2 | 10.1 | 69.7 | 0.7 | 0.4 |
| | 0.5 | 9.6 | 20 | 1.4 | 3.4 | 1.8 | 1.8 | 0.0 | 0.0 |
| | 0.5 | 7.4 | 100 | 8.0 | 21.5 | 0.3 | 0.0 | 0.0 | 0.0 |
| | 0.5 | 8.4 | 100 | 4.8 | 3.3 | 1.6 | 0.7 | 0.0 | 0.0 |
| | 0.5 | 9.6 | 100 | 2.4 | 3.2 | 4.2 | 8.7 | 0.0 | 0.0 |

| | | | |
|---|---|---|---|
| Tissue: | Kidney | Breast | Breast |
| Cell Line: | 293T | MCF7 | MCF7 |
| Block: | 201022A | 191105A | 191105A |
| Replicates: | two | three | three |

TABLE 2

| | Decrosslink | Lysis | Test cpd. | Compound 3 | | Compound 4 | | Compound 5 | |
|---|---|---|---|---|---|---|---|---|---|
| | Time (hr) | buffer pH | conc. (mM) | R | T | R | T | R | T |
| Pos. Control | 4 | 7.4 | 0 | 408.4 | 1137.1 | 493.0 | 1746.2 | 778.5 | 1032.7 |
| Neg. Control | 0.5 | 7.4 | 0 | 52.5 | 36.0 | 31.2 | 29.2 | 34.6 | 13.6 |
| | 0.5 | 7.4 | 20 | 379.5 | 1142.6 | 400.6 | 1361.0 | 563.9 | 1217.1 |
| | 0.5 | 8.4 | 20 | 153.3 | 280.0 | 294.6 | 843.6 | 148.8 | 71.0 |
| | 0.5 | 9.6 | 20 | 65.5 | 85.3 | 63.4 | 155.1 | 69.3 | 34.3 |
| | 0.5 | 7.4 | 100 | 34.2 | 10.9 | 8.1 | 0.6 | 0.0 | 0.0 |
| | 0.5 | 8.4 | 100 | 112.5 | 104.4 | 66.1 | 29.7 | 54.9 | 0.0 |
| | 0.5 | 9.6 | 100 | 214.4 | 445.3 | 97.4 | 275.9 | 165.8 | 140.8 |
| Pos. Control | 4 | 7.4 | 0 | 7.8 | 31.6 | 15.8 | 59.8 | 22.5 | 76.2 |
| Neg. Control | 0.5 | 7.4 | 0 | 1.0 | 1.0 | 1.0 | 1.0 | 1.0 | 1.0 |
| | 0.5 | 7.4 | 20 | 7.2 | 31.7 | 12.9 | 46.6 | 16.3 | 89.8 |
| | 0.5 | 8.4 | 20 | 2.9 | 7.8 | 9.5 | 28.9 | 4.3 | 5.2 |
| | 0.5 | 9.6 | 20 | 1.2 | 2.4 | 2.0 | 5.3 | 2.0 | 2.5 |
| | 0.5 | 7.4 | 100 | 0.7 | 0.3 | 0.3 | 0.0 | 0.0 | 0.0 |
| | 0.5 | 8.4 | 100 | 2.1 | 2.9 | 2.1 | 1.0 | 1.6 | 0.0 |
| | 0.5 | 9.6 | 100 | 4.1 | 12.4 | 3.1 | 9.5 | 4.8 | 10.4 |

| | | | |
|---|---|---|---|
| Tissue: | Breast | Breast | Breast |
| Cell Line: | MCF7 | MCF7 | MCF7 |
| Block: | 131014A | 210615A | 191105A |
| Replicates: | three | three | three |

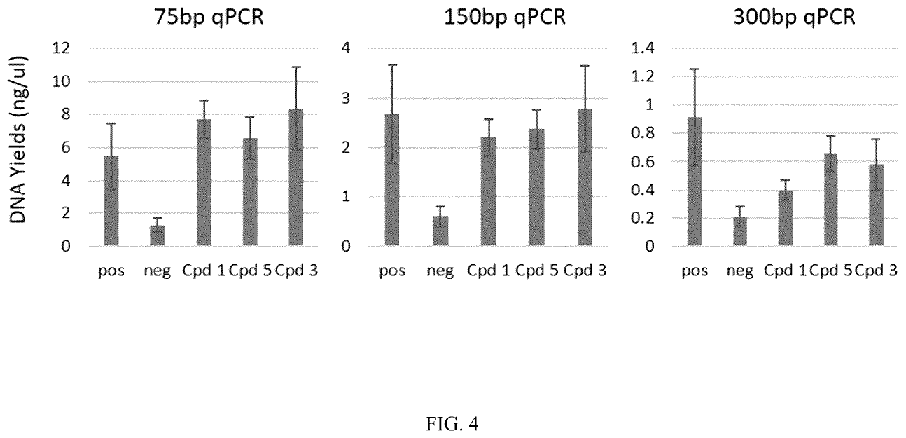

The quality of DNA decrosslinked by these leading candidate was further accessed by testing on FFPE tissues from human donors using Next Generation Sequencing (NGS). Healthy colon FFPE donor tissue underwent rapid preprocessing using Compounds 1, 3, and 5 (Cpd. 1, Cpd. 3, and Cpd. 5, respectively) with decrosslinking carried out at 80° C. for 30 minutes along with positive controls (80° C. for 4 hours) and negative controls (80° C. for 30 minutes). DNA was then purified on the Maxwell® RSC 48 instrument (Promega Corp). DNA quantity and fragment sizes were assessed using Promega's ProNex® DNA QC Assay, which amplifies 75 bp, 150 bp, and 300 bp qPCR amplicons. Amplifiable DNA quantity and fragment sizes from samples rapidly decrosslinked with Cpd 1, Cpd 5, and Cpd 3 reactions were comparable with positive control samples (FIG. 4). Averages of three purification replicates are plotted with error bars representing standard deviation.

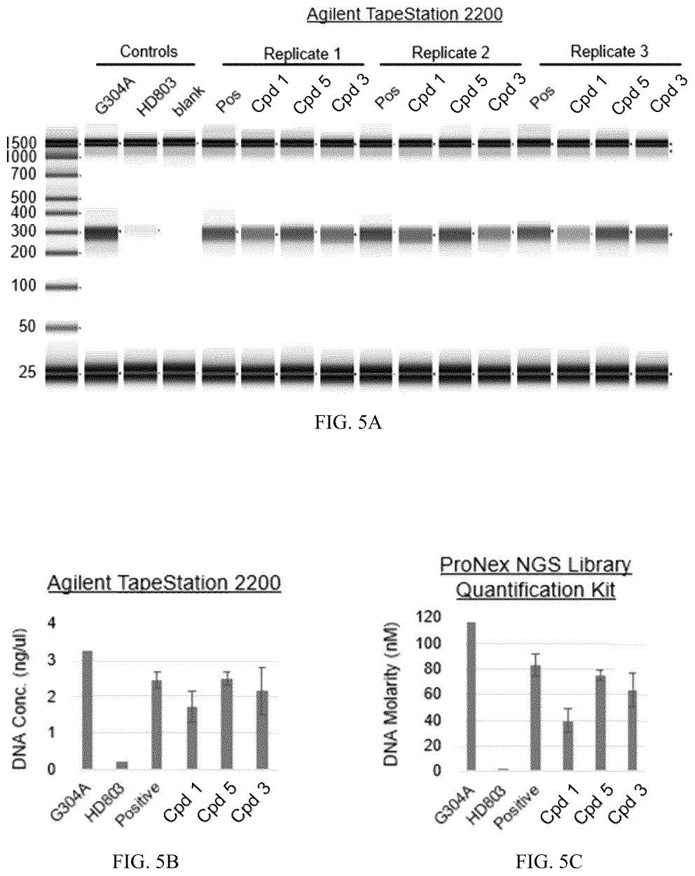

NGS libraries were constructed using Illumina's AmpliSeq for Cancer HotSpot Panel v2. FIG. 5A shows molecular weight of libraries as determined by electrophoresis with Agilent TapeStation. FIG. 5B shows quantification of electrophoresed libraries. FIG. 5C shows quantity of libraries as determined by qPCR with ProNex® NGS Library Quantification Kit. G304A is high quality human gDNA pooled from multiple donors (Promega Cat. No. G3041). HD803 is formalin-damaged human gDNA pooled from multiple immortalized cell lines for use as a Multiplex Reference Standard (Horizon, HD803). NGS libraries from rapid decrosslinking reactions were of expected size and sufficient quantity for sequencing.

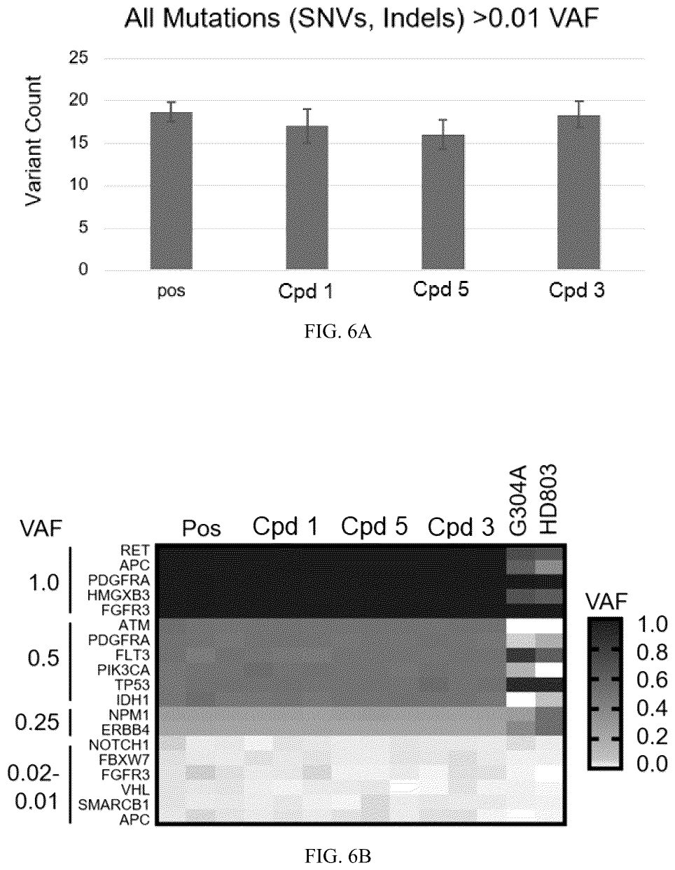

Libraries were sequenced using paired-end 150-cycle run on an Illumina MiSeq. Sequence Analysis Viewer and FastQC was used to assess sequencing performance. Error rate, % Q30, cluster density, cluster % PF, Phas/Prephas (%) were all within expected ranges (data not shown). Bioinformatic analyses was performed with llumina's BaseSpace DNA Amplicon App. Single-nucleotide variants (SNVs) and insert-deletions (Indels) with variant allele frequencies (VAF) above 1% were called. Total counts were comparable between positive control and rapid decrosslinking catalyst purifications (FIG. 6A). VAFs of mutations identified in the positive control were found to be highly concordant with catalyst purifications (FIG. 6B). This indicates that rapidly decrosslinked DNA maintains high sequence quality, allowing for NGS to be used to make VAF calls near the limit-of-detection.

### Example 2

### Decrosslinking Time Course

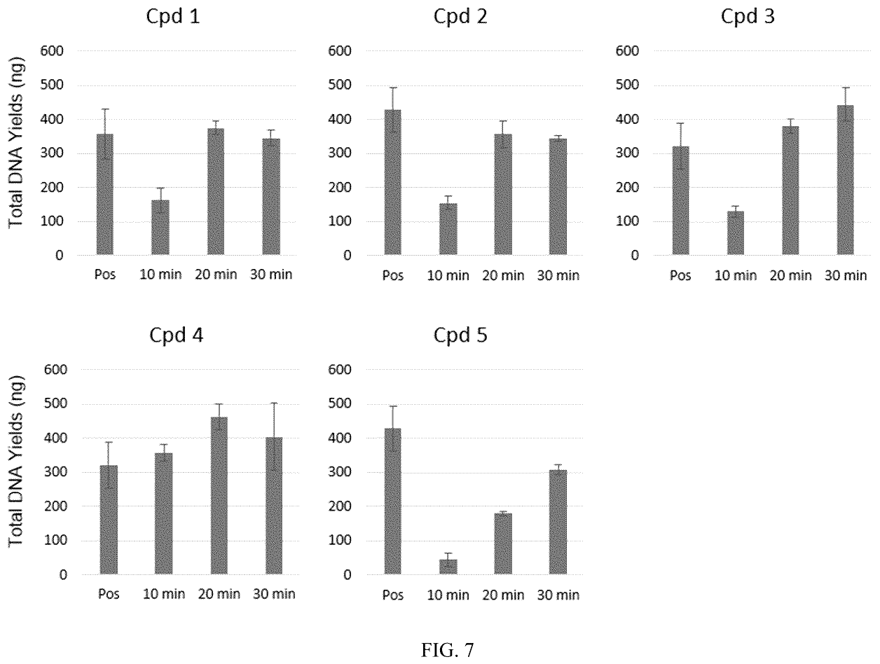

A time course experiment was conducted to determine the minimal decrosslinking time needed for complete decrosslinking with catalyst compounds (FIG. 7). Cell pellet artificial FFPE (Amsbio LLC) were decrosslinked with Cpd 1-5 at 80° C. for 10, 20, and 30 minutes. Amplifiable DNA yields were accessed using a PrimeTime qPCR Assay targeting a 102 bp amplicon in the RPPH1 gene (IDT). Minimal decrosslinking times ranged from 10 minutes (Cpd 4), 20 minutes (Cpd 1, Cpd 2, Cpd 3), and 30 minutes (Cpd 5). DNA yields were compared to a positive control (Pos) decrosslinked at 80° C. for 4 hours without catalysts.

### Example 3

### pH Optimization

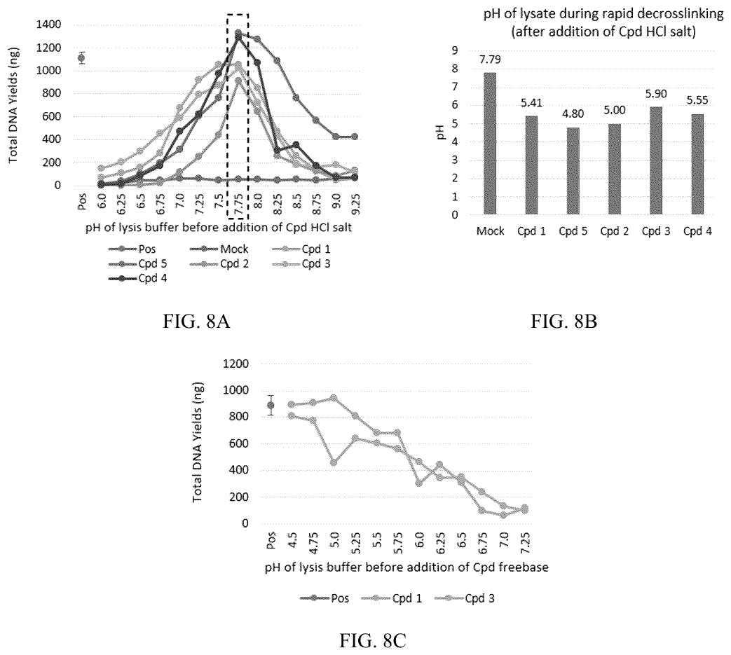

To determine the optimal pH for rapid decrosslinking, cell pellet artificial FFPE (Amsbio LLC) were decrosslinked at 80° C. for 30 minutes with 20 mM of Cpd 1-5 HCl salts in a range of pH lysis buffers (FIG. 8A). Amplifiable DNA yields were accessed using a PrimeTime qPCR Assay targeting a 164 bp amplicon in the TERT gene (IDT). DNA yields were compared to a positive control (Pos) decrosslinked in pH 8 lysis buffer at 80° C. for 4 hours without catalysts, and a negative control (mock) decrosslinked in a range of pH lysis buffers at 80° C. for 30 minutes without catalysts. To determine the working pH of the lysate during rapid decrosslinking, 20 mM of Cpd HCl salts were added to lysis buffer pH 7.75 (optimum found in FIG. 8A). Optimal pH fell between 4.80-5.90 (FIG. 8B). To confirm the pH requirement can be achieved through the buffer, an experiment was performed using catalyst freebases (FIG. 8C). After Proteinase K digestion, pH of lysates were lowered with pre-determined amounts of HCl to create a range of pH lysis buffers. 20 mM of freebase Cpd 1 and Cpd 3 were added to the lysate and decrosslinked for 30 minutes at 80° C. Amplifiable DNA yields were accessed using a PrimeTime qPCR Assay targeting a 164 bp amplicon in the TERT gene (IDT). DNA yields peaked in buffers with pH 4.5-6.0.

### Example 4

### Catalyst Concentration Optimization

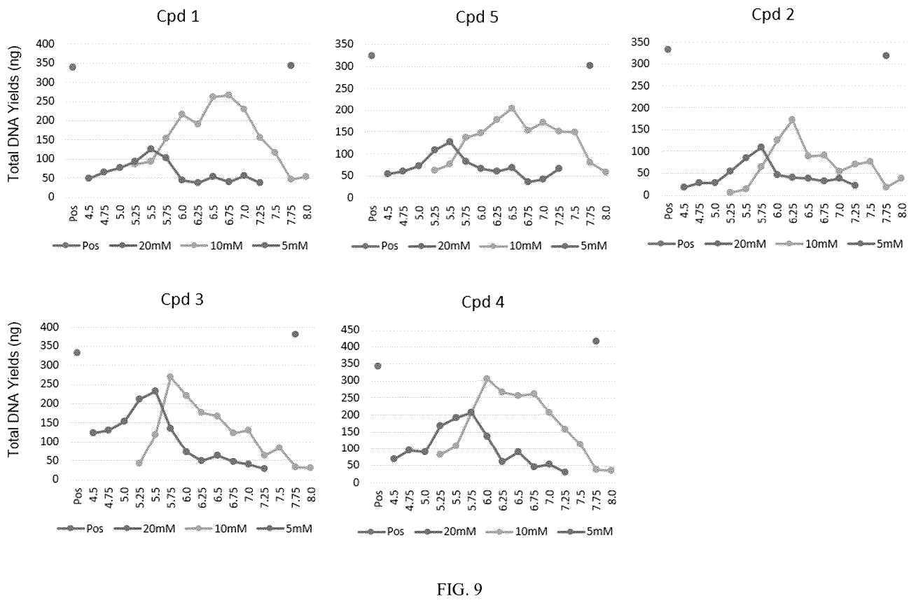

Optimal concentration of decrosslinking catalyst compounds were determined by adding 20 mM, 10 mM, and 5 mM of Cpd HCl 1-5 and decrosslinking at 80° C. for 30 minutes (FIG. 9). A range of pH lysis buffers were used to accommodate for less HCl (from the Cpd HCl salt) being added. Amplifiable DNA yields were accessed using a PrimeTime qPCR Assay targeting a 102 bp amplicon in the RPPH1 gene (IDT). DNA yields were compared to a positive control (Pos) decrosslinked in pH 8 lysis buffer at 80° C. for 4 hours without catalysts. Amplifiable DNA yields were found to be concentration dependent.

### Example 5

### Protease Compatibility Screening

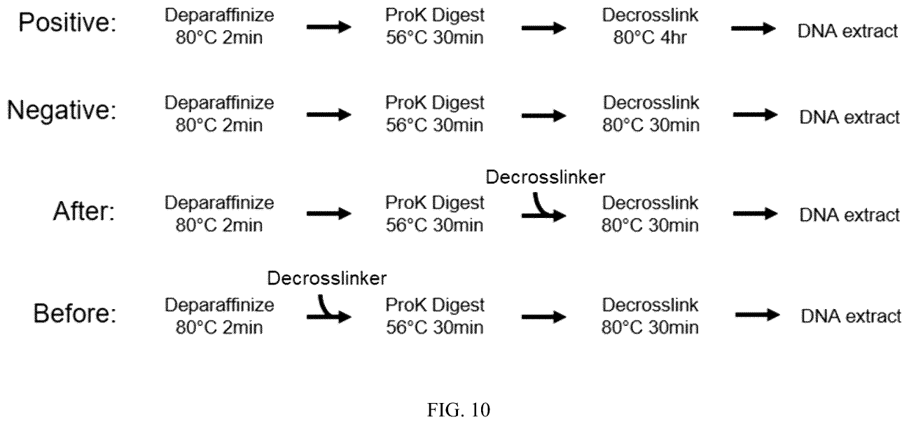

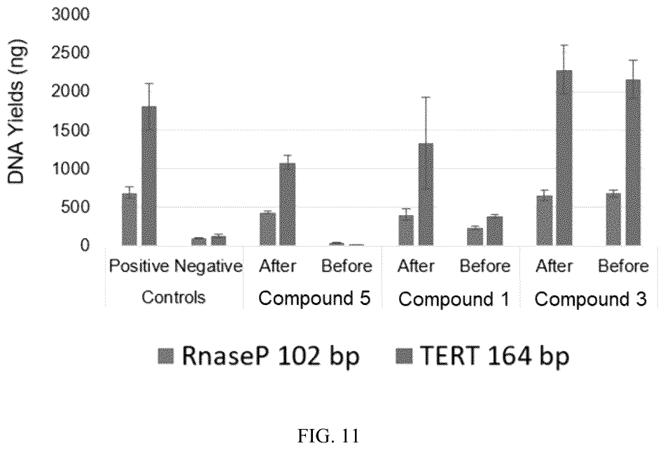

After identification of candidate decrosslinking compounds, a secondary screen was conducted to confirm compatibility of the compounds with ProK. General screening protocols, including positive and negative controls, are shown in FIG. 10. Decrosslinking catalysts were included in the lysis buffer mastermix and added before the ProK digest incubation. The effects on DNA yields were compared to samples wherein decrosslinking compounds were added after the ProK digest, a positive control (4 hour decrosslink without any decrosslinking catalyst), and a negative control (30 minute decrosslink without any decrosslinking catalyst). Data are shown in FIG. 11. Results on yields differed between nucleophiles. Compound 3 and to a lesser degree Compound 1, added before the ProK digestion, showed enhanced DNA yields compared to the negative control. This indicates that the ProK digestion and nucleophilic activity of Compounds 1 and 3 did not negatively impact one another.

### Example 6

### Assessment of Incubation Time

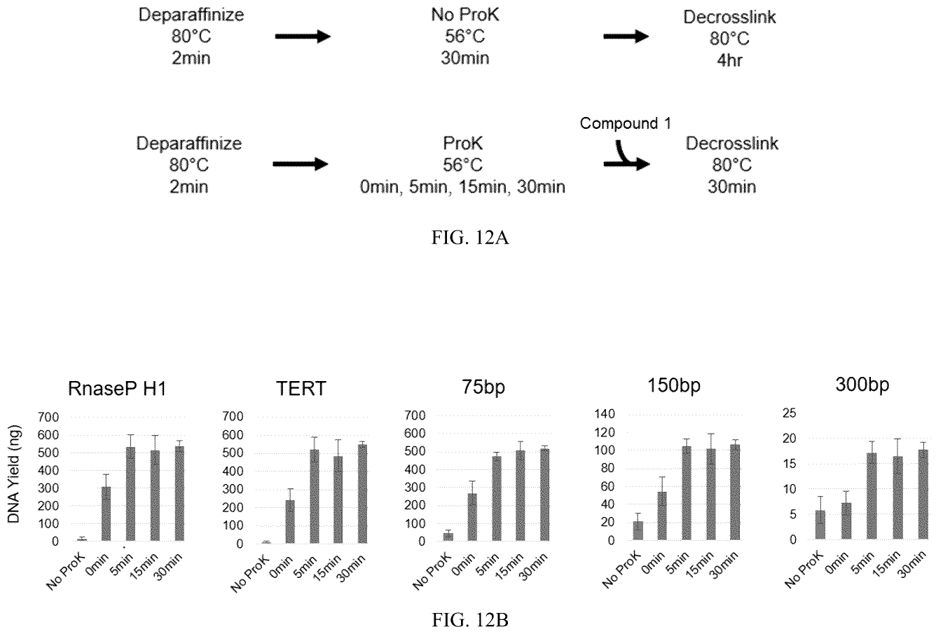

ProK is a robust and stable enzyme, however, at temperatures above 60° C., it rapidly loses activity. To assess the effect of incubation time on ProK digestion, samples were digested for 0 min, 5 min, 15 min, and 30 min and then decrosslinked with Compound 1 at 80° C. for 30 minutes (see FIG. 12A), and then total amplifiable DNA yields (ng) was assessed by a duplex qPCR assay and the ProNEX® QC qPCR assay (75 bp, 150 bp, and 300 bp amplicons).

Results are shown in FIG. 12B. A 5 min incubation at 56° C. yielded high levels of amplifiable DNA. Interestingly, a 0 min (i.e., straight to 80° C.) incubation yielded high levels of DNA compared to a no ProK control. This suggests that ProK digestion may have occurred while mixing the lysis buffer with the tissue (˜3 min at room temperature) and while the lysate ramped to 80° C. for decrosslinking (˜5 min at 21° C.-80° C.).

### Example 7

### Single-Step Decrosslinking

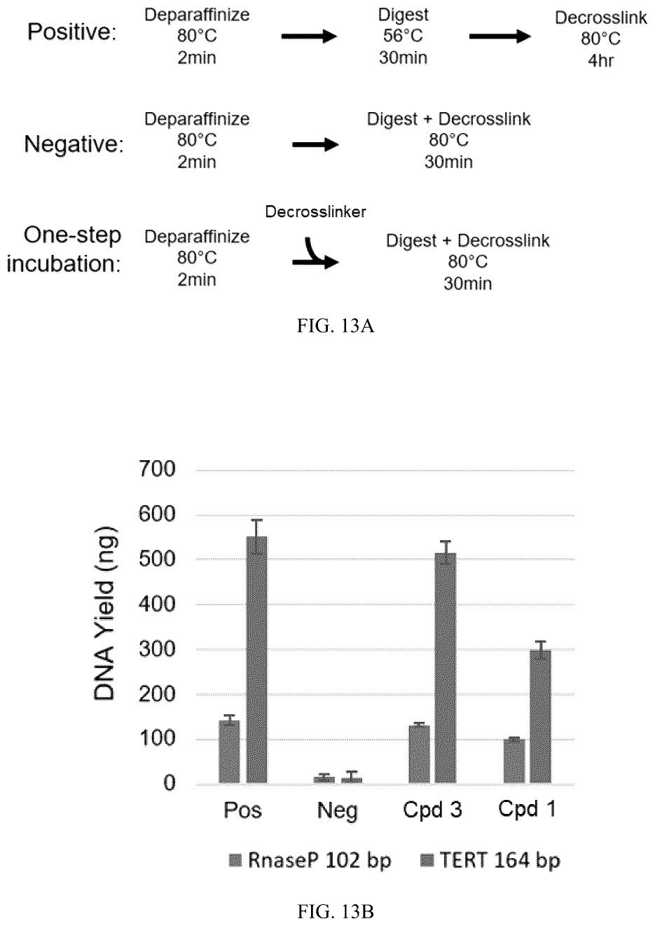

Having identified decrosslinking compounds that are compatible with ProK (FIG. 11) and allow for rapid ProK digestion (FIG. 12B), a single step assay was used to determine if it was sufficient for high FFPE DNA yields (see FIG. 13A). Deparaffinized FFPE cells were given a mastermix of MC118 lysis buffer (Promega Corp.), ProK solution, and 20 mM of Compound 1 or Compound 3. Lysates were incubated at 80° C. for 30 minutes, DNA purified on a Maxwell® RSC instrument, and amplifiable DNA assessed using qPCR (FIG. 13B). Both Compounds 1 and 3 yielded DNA levels similar to a positive control standard two-step protocol. Accordingly, these compounds permit one-step incubations for the preprocessing of FFPE tissues and subsequent nucleic acid purification.

### Example 8

Catalyst-Decrosslinked FFPE DNA Purified from a Variety of Human Tissues and Assessed by Amplification, Dye-Binding, and Absorbance

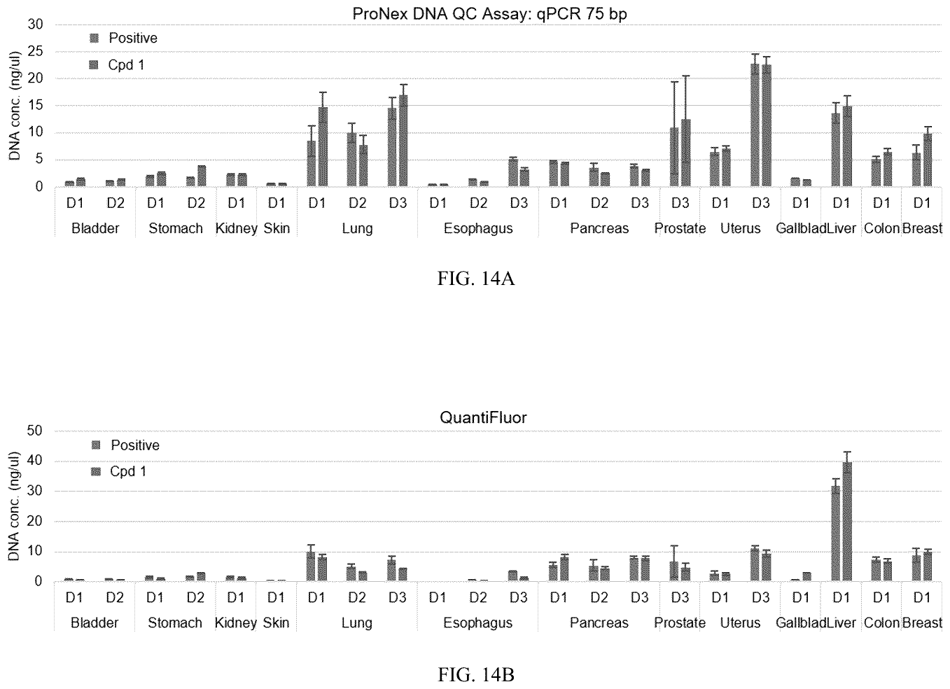

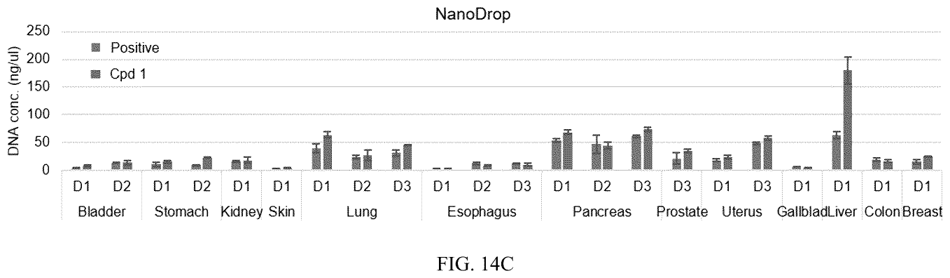

Catalysts were tested in 13 human FFPE tissues (FIGS. 14A-14C: bladder, stomach, kidney, skin, lung, esophagus, pancreas, prostate, uterus, gallbladder, liver, colon, and breast). 30 mM compound 1 (Cpd 1) was added to each FFPE lysate and decrosslinked at 80° C. for 30 minutes (Cpd 1) and compared to a mock no compound treatment decrosslinked at 80° C. for 4 hours (Positive). One to three donors per tissue, and two to four purification replicates per donor were performed. DNA yields were assessed by RT-qPCR using Promega ProNex® DNA QC Assay (Cat. No. NG1002) (FIG. 14A), fluorescent dsDNA dye using Promega QuantiFluor® dsDNA System (Cat. No. E2671) (FIG. 14B), and absorbance using a ThermoFisher NanoDrop (FIG. 14C).

1. A method of decrosslinking a formaldehyde cross-linked biological sample, comprising a step of simultaneously contacting the sample with a protease and a decrosslinking catalyst, wherein the decrosslinking catalyst is a compound of formula (I), formula (II), or formula (III);
wherein the compound of formula (I) is: 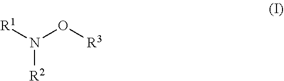
or a salt thereof,
wherein:
R¹ is selected from H and C₁-C₆ alkyl;
R² is selected from H, C₁-C₆ alkyl, aryl, heteroaryl, aryl-C₁-C₄-alkyl, and heteroaryl-C₁-C₄-alkyl;
R³ is selected from H, C₁-C₆ alkyl, and —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or more substituents independently selected from halo, C₁-C₄ alkyl, hydroxy, C₁-C₄ alkoxy, —NR^{a1}R^{b1}, —C(O)NR^{c1}R^{d1}, —COOR^{e1}, and —SO₂NR^{f1}R^{g1};
wherein each alkyl is unsubstituted or substituted with 1 or more substituents independently selected from halo, hydroxy, C₁-C₄ alkoxy, thiol, C₁-C₄ alkylthio, —NR^{a1}R^{b1}, —C(O)NR^{c1}R^{d1}, —COOR^{e1}, —SO₂NR^{f1}R^{g1}, —ONR^{h1}Rⁱ¹, —NR^{j1}OR^{k1}, —PO₃H₂, —SO₃H, optionally substituted C₃-C₆ cycloalkyl, and optionally substituted 3- to 6-membered heterocyclyl; and
wherein R^{a1}, R^{b1}, R^{c1}, R^{d1}, R^{e1}, R^{f1}, R^{g1}, R^{h1}, Rⁱ¹, R^{j1}, and R^{k1} are each independently selected from H, C₁-C₄ alkyl, C₁-C₄ hydroxyalkyl, and C₁-C₄ carboxyalkyl;
wherein R¹ and R², together with the nitrogen atom to which they are attached, are optionally taken together to form an optionally substituted 4- to 8-membered ring; and
wherein R² and R³, together with the atoms to which they are attached, are taken together to form an optionally substituted 4- to 8-membered ring;
wherein the compound of formula (II) is:
A—NR^{x}R^{y} (II)
or a salt thereof, wherein:
A is selected from H, C₁-C₆-alkyl, aryl, heteroaryl, C₃-C₆-cycloalkyl, aryl-C₁-C₄-alkyl, heteroaryl-C₁-C₄-alkyl, and —Q—R⁵;
Q is selected from —NR^{a2}—, —NR^{b2}CO—, —SO₂—, —SO₂NR^{c2}—, and —NR^{d2}COCONR^{e2}NR^{f2}—;
R⁵ is selected from H, C₁-C₆-alkyl, aryl, heteroaryl, and C₃-C₆-cycloalkyl;
R^{a2}, R^{b2}, R^{e2}, R^{d2}, R^{e2}, and R^{f2} are each independently selected from H and C₁-C₆ alkyl;
R^{x} and R^{y} are each independently selected from H and C₁-C₆ alkyl; or R^{x} and R^{y}, together with the nitrogen atom to which they are attached, form an optionally substituted 4- to 8-membered ring;
wherein each aryl or heteroaryl is unsubstituted or substituted independently selected from halo, hydroxyl, C₁-C₄ alkyl, C₁-C₄ alkoxy, C₁-C₄ hydroxyalkyl, —P(O)(OH)₂, —B(OH)₂, —COOR^{g2}, —CONR^{h2}Rⁱ², —SO₂NR^{j2}R^{k2}, and —SO₂OR^{l2}; and
wherein each alkyl or cycloalkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOR^{g2}, —PO₃H₂, —SO₂OR^{l2}, and —NR^{m2}Rⁿ²;
wherein each R^{g2}, R^{h2}, Rⁱ², R^{j2}, R^{k2}, R^{l2}, R^{m2}, and Rⁿ² is independently selected from H and methyl;
wherein the compound of formula (III) is:
B—X (III)
or a salt thereof, wherein:
B is aryl, heteroaryl, C₁-C₆ alkyl, aryl-C₁-C₄-alkyl-, aryl-C₂-C₄-alkoxy-, heteroaryl-C₁-C₄-alkyl-, and heteroaryl-C₂-C₄-alkoxy-; and
X is selected from —COOH, —PO₃H₂, —B(OH)₂, and —SO₃H;
wherein each aryl and heteroaryl is independently unsubstituted or substituted with 1 or more substituents independently selected from halo, C₁-C₄ alkyl, C₁-C₄ alkoxy, hydroxy, C₁-C₄ hydroxyalkyl, —NR^{a3}R^{b3}, —COOR^{c3}, —SO₃R^{d3}, —PO₃H₂, and —B(OH)₂;
wherein each alkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOH, —PO₃H₂ and —SO₃H; and
wherein R^{a3}, R^{b3}, R^{c3}, and R^{d3} are each independently selected from H, —CH₃, and —CH₂CH₃.

2. The method of claim 1, wherein the decrosslinking catalyst is a compound of formula (I), or a salt thereof.

3.-5. (canceled)

6. The method of claim 2, wherein the compound of formula (I) is selected from: 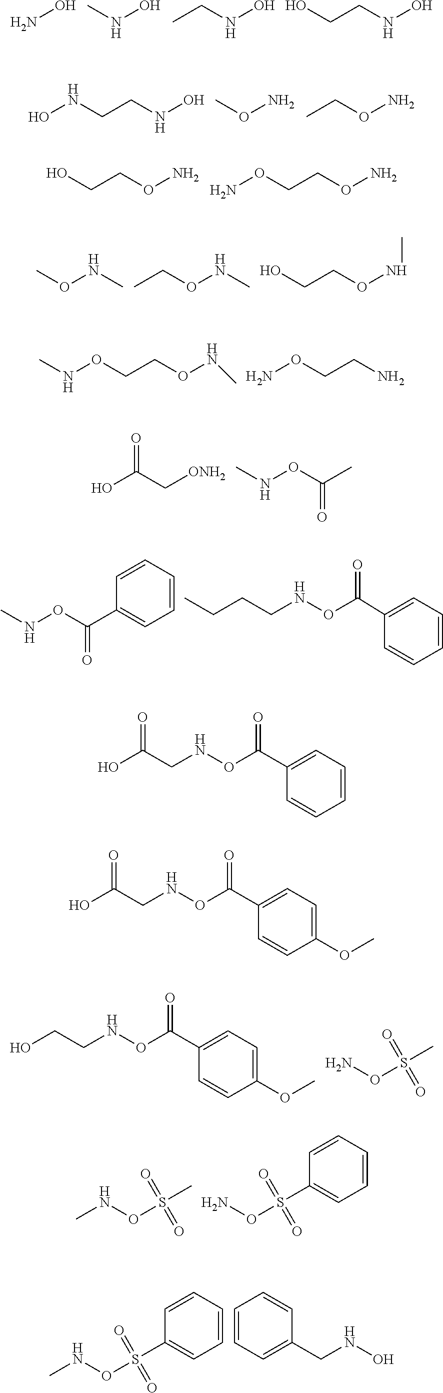 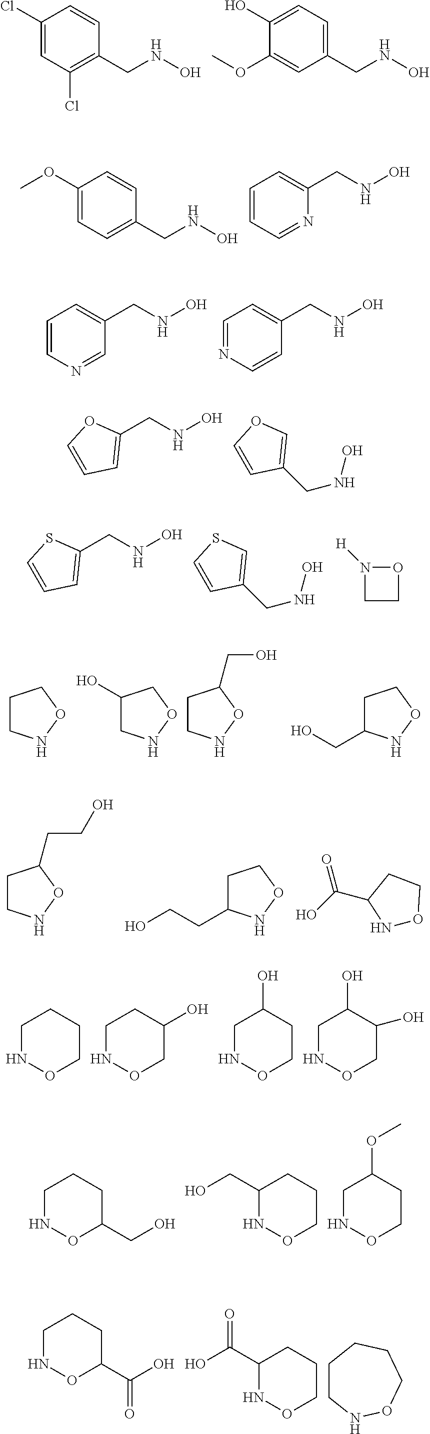 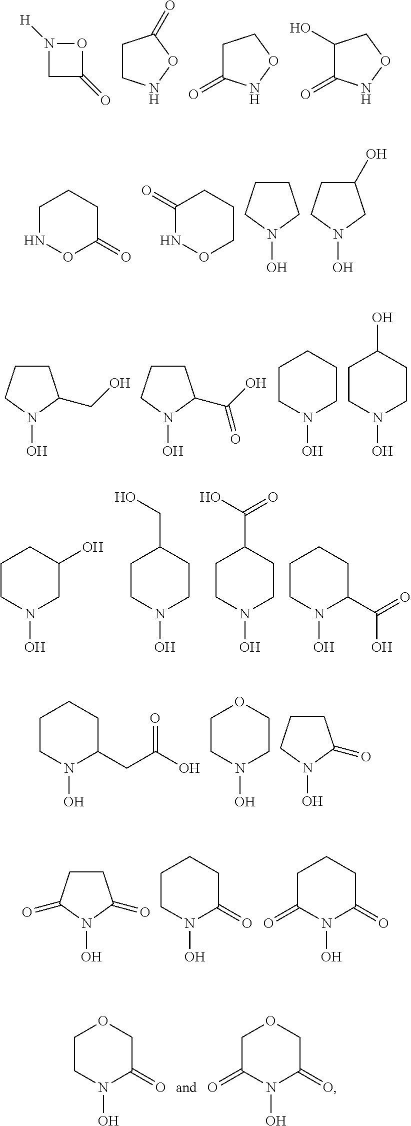
and salts thereof.

7. The method of claim 1, wherein the decrosslinking catalyst is a compound of formula (II), or a salt thereof.

8.-11. (canceled)

12. The method of claim 7, wherein the compound of formula (II) is selected from: 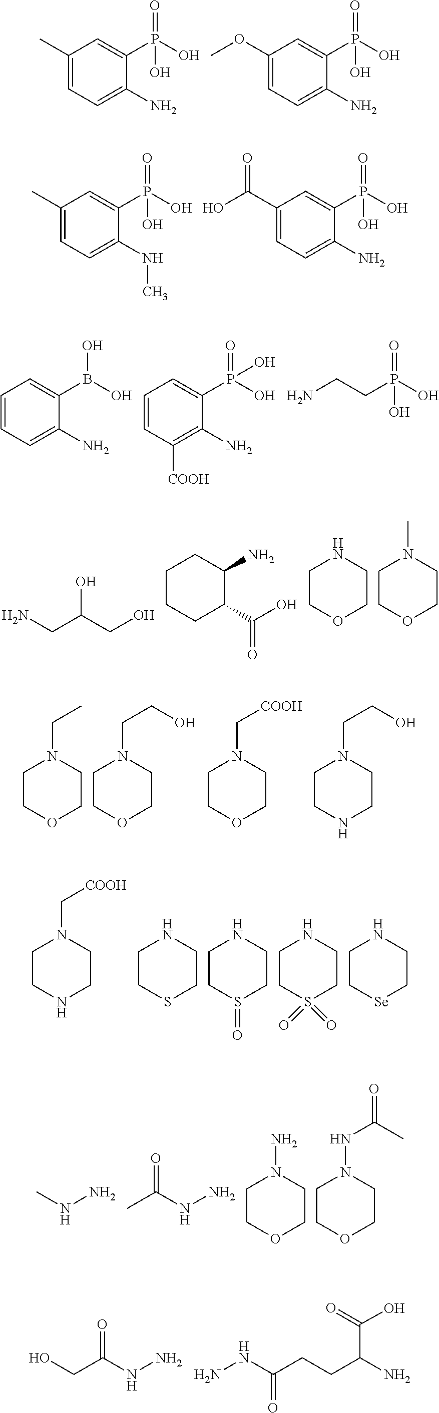 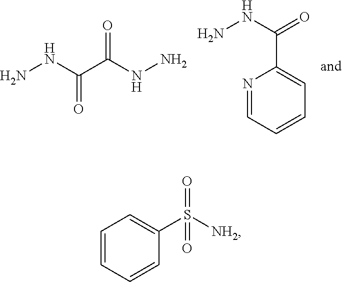 [SMILES: NNC(=O)C(=O)NN ; NNC(=O)c1ccccn1 ; NS(=O)(=O)c1ccccc1]
and salts thereof.

13. The method of claim 1, wherein the decrosslinking catalyst is a compound of formula (III), or a salt thereof.

14.-15. (canceled)

16. The method of claim 13, wherein the compound of formula (II) is selected from: 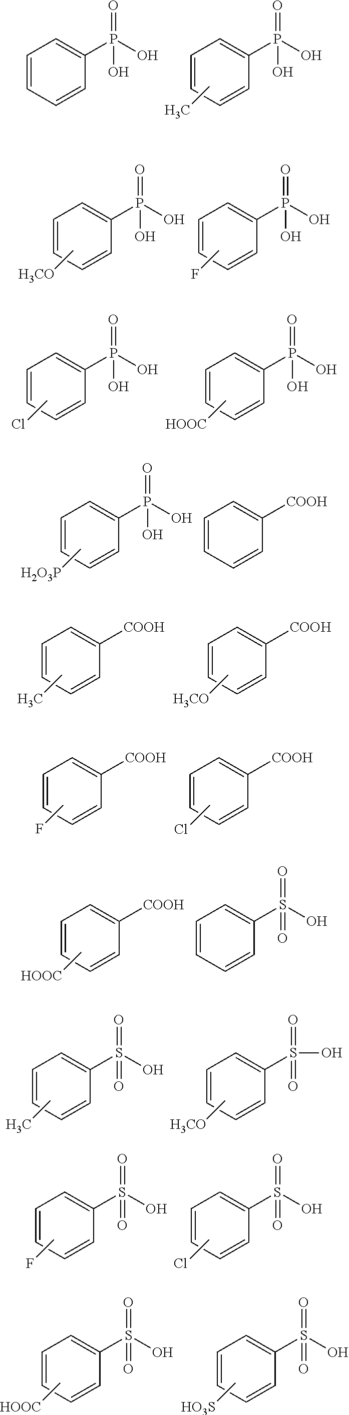 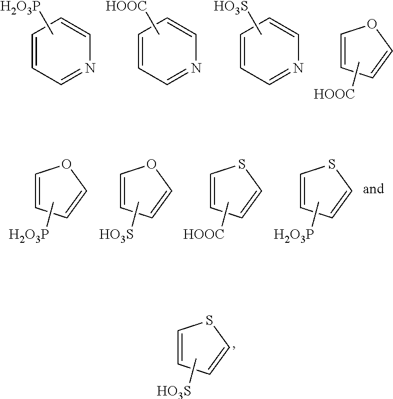
and salts thereof.

17. The method of claim 1, wherein the decrosslinking catalyst is selected from: 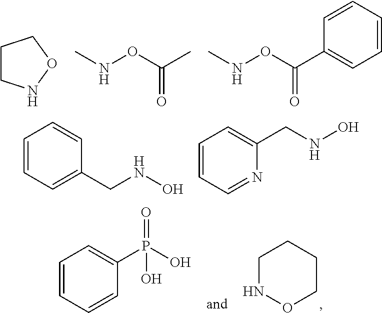
and salts thereof.

18. The method of claim 1, wherein the decrosslinking catalyst is in the form of a salt.

19. The method of claim 1, wherein the decrosslinking catalyst is in the form of a hydrochloric acid salt.

20. The method of claim 1, wherein the method comprises contacting the sample with effective amounts of at least two different decrosslinking catalysts, or salts thereof.

21.-22. (canceled)

23. The method of claim 1, wherein the sample is a formalin-fixed paraffin embedded tissue sample, and the method further comprises a step of deparaffinizing the sample prior to contacting the sample with the protease and the decrosslinking catalyst.

24. (canceled)

25. The method of claim 1, wherein the protease is proteinase K.

26. The method of claim 1, wherein the contacting step is conducted for about 5 minutes to about 120 minutes, and a temperature of about 20° C. to about 100° C.

27.-41. (canceled)

42. The method of claim 1, further comprising extracting one or more components from the sample after the contacting step.

43. The method of claim 42, wherein the one or more components are selected from nucleic acids and proteins.

44. The method of claim 43, wherein the one or more components are nucleic acids, and the method further comprises a step of detecting and/or amplifying one or more nucleic acids.

45. The method of claim 44, further comprising a step selected from dye binding, absorbance, and enzymatic digestion.

46. A composition comprising a protease and a decrosslinking catalyst, wherein the decrosslinking catalyst is a compound of formula (I), formula (II), or formula (III); wherein the compound of formula (I) is: 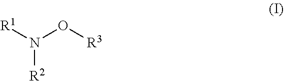
or a salt thereof,
wherein:
R¹ is selected from H and C₁-C₆ alkyl;
R² is selected from H, C₁-C₆ alkyl, aryl, heteroaryl, aryl-C₁-C₄-alkyl, and heteroaryl-C₁-C₄-alkyl;
R³ is selected from H, C₁-C₆ alkyl, and —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or more substituents independently selected from halo, C₁-C₄ alkyl, hydroxy, C₁-C₄ alkoxy, —NR^{a1}R^{b1}, —C(O)NR^{c1}R^{d1}, —COOR^{e1}, and —SO₂NR^{f1}R^{g1};
wherein each alkyl is unsubstituted or substituted with 1 or more substituents independently selected from halo, hydroxy, C₁-C₄ alkoxy, thiol, C₁-C₄ alkylthio, —NR^{a1}R^{b1}, —C(O)NR^{c1}R^{d1}, —COOR^{e1}, —SO₂NR^{f1}R^{g1}, —ONR^{h1}Rⁱ¹, —NR^{j1}OR^{k1}, —PO₃H₂, —SO₃H, optionally substituted C₃-C₆ cycloalkyl, and optionally substituted 3- to 6-membered heterocyclyl; and
wherein R^{a1}, R^{b1}, R^{c1}, R^{d1}, R^{e1}, R^{f1}, R^{g1}, R^{h1}, Rⁱ¹, R^{j1}, and R^{k1} are each independently selected from H, C₁-C₄ alkyl, C₁-C₄ hydroxyalkyl, and C₁-C₄ carboxyalkyl;
wherein R¹ and R², together with the nitrogen atom to which they are attached, are optionally taken together to form an optionally substituted 4- to 8-membered ring; and
wherein R² and R³, together with the atoms to which they are attached, are taken together to form an optionally substituted 4- to 8-membered ring;
wherein the compound of formula (II) is:
A—NR^{x}R^{y} (II)
or a salt thereof, wherein:
A is selected from H, C₁-C₆-alkyl, aryl, heteroaryl, C₃-C₆-cycloalkyl, aryl-C₁-C₄-alkyl, heteroaryl-C₁-C₄-alkyl, and —Q—R⁵;
Q is selected from —NR^{a2}—, —NR^{b2}CO—, —SO₂—, —SO₂NR^{c2}—, and —NR^{d2}COCONR^{e2}NR^{f2}—;
R⁵ is selected from H, C₁-C₆-alkyl, aryl, heteroaryl, and C₃-C₆-cycloalkyl;
R^{a2}, R^{b2}, R^{e2}, R^{d2}, R^{e2}, and R^{f2} are each independently selected from H and C₁-C₆ alkyl;
R^{x} and R^{y} are each independently selected from H and C₁-C₆ alkyl; or R^{x} and R^{y}, together with the nitrogen atom to which they are attached, form an optionally substituted 4- to 8-membered ring;
wherein each aryl or heteroaryl is unsubstituted or substituted independently selected from halo, hydroxyl, C₁-C₄ alkyl, C₁-C₄ alkoxy, C₁-C₄ hydroxyalkyl, —P(O)(OH)₂, —B(OH)₂, —COOR^{g2}, —CONR^{h2}Rⁱ², —SO₂NR^{j2}R^{k2}, and —SO₂OR^{l2}; and
wherein each alkyl or cycloalkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOR^{g2}, —PO₃H₂, —SO₂OR^{l2}, and —NR^{m2}Rⁿ²;
wherein each R^{g2}, R^{h2}, Rⁱ², R^{j2}, R^{k2}, R^{l2}, R^{m2}, and Rⁿ² is independently selected from H and methyl;
wherein the compound of formula (III) is:
B—X (III)
or a salt thereof, wherein:
B is aryl, heteroaryl, C₁-C₆ alkyl, aryl-C₁-C₄-alkyl-, aryl-C₂-C₄-alkoxy-, heteroaryl-C₁-C₄-alkyl-, and heteroaryl-C₂-C₄-alkoxy-; and
X is selected from —COOH, —PO₃H₂, —B(OH)₂, and —SO₃H;
wherein each aryl and heteroaryl is independently unsubstituted or substituted with 1 or more substituents independently selected from halo, C₁-C₄ alkyl, C₁-C₄ alkoxy, hydroxy, C₁-C₄ hydroxyalkyl, —NR^{a3}R^{b3}, —COOR^{c3}, —SO₃R^{d3}, —PO₃H₂, and —B(OH)₂;
wherein each alkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOH, —PO₃H₂ and —SO₃H; and
wherein R^{a3}, R^{b3}, R^{c3}, and R^{d3} are each independently selected from H, —CH₃, and —CH₂CH₃.

47.-75. (canceled)

76. A kit for decrosslinking a formaldehyde cross-linked biological sample, the kit comprising: (i) a decrosslinking catalyst, wherein the decrosslinking catalyst is a compound of formula (I), formula (II), or formula (III); and (ii) instructions for decrosslinking a formaldehyde cross-linked biological sample, instructing a user to simultaneously contact the sample with the decrosslinking catalyst and a protease;
wherein the compound of formula (I) is: 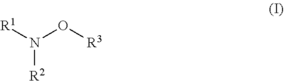
or a salt thereof,
wherein:
R¹ is selected from H and C₁-C₆ alkyl;
R² is selected from H, C₁-C₆ alkyl, aryl, heteroaryl, aryl-C₁-C₄-alkyl, and heteroaryl-C₁-C₄-alkyl;
R³ is selected from H, C₁-C₆ alkyl, and —X—R⁴, wherein X is selected from —C(O)— and —SO₂—, and R⁴ is selected from C₁-C₆-alkyl, aryl, and heteroaryl;
wherein each aryl and heteroaryl is independently unsubstituted or substituted with 1 or more substituents independently selected from halo, C₁-C₄ alkyl, hydroxy, C₁-C₄ alkoxy, —NR^{a1}R^{b1}, —C(O)NR^{c1}R^{d1}, —COOR^{e1}, and —SO₂NR^{f1}R^{g1};
wherein each alkyl is unsubstituted or substituted with 1 or more substituents independently selected from halo, hydroxy, C₁-C₄ alkoxy, thiol, C₁-C₄ alkylthio, —NR^{a1}R^{b1}, —C(O)NR^{c1}R^{d1}, —COOR^{e1}, —SO₂NR^{f1}R^{g1}, —ONR^{h1}Rⁱ¹, —NR^{j1}OR^{k1}, —PO₃H₂, —SO₃H, optionally substituted C₃-C₆ cycloalkyl, and optionally substituted 3- to 6-membered heterocyclyl; and
wherein R^{a1}, R^{b1}, R^{c1}, R^{d1}, R^{e1}, R^{f1}, R^{g1}, R^{h1}, Rⁱ¹, R^{j1}, and R^{k1} are each independently selected from H, C₁-C₄ alkyl, C₁-C₄ hydroxyalkyl, and C₁-C₄ carboxyalkyl;
wherein R¹ and R², together with the nitrogen atom to which they are attached, are optionally taken together to form an optionally substituted 4- to 8-membered ring; and
wherein R² and R³, together with the atoms to which they are attached, are taken together to form an optionally substituted 4- to 8-membered ring;
wherein the compound of formula (II) is:
A—NR^{x}R^{y} (II)
or a salt thereof, wherein:
A is selected from H, C₁-C₆-alkyl, aryl, heteroaryl, C₃-C₆-cycloalkyl, aryl-C₁-C₄-alkyl, heteroaryl-C₁-C₄-alkyl, and —Q—R⁵;
Q is selected from —NR^{a2}—, —NR^{b2}CO—, —SO₂—, —SO₂NR^{c2}—, and —NR^{d2}COCONR^{e2}NR^{f2}—;
R⁵ is selected from H, C₁-C₆-alkyl, aryl, heteroaryl, and C₃-C₆-cycloalkyl;
R^{a2}, R^{b2}, R^{e2}, R^{d2}, R^{e2}, and R^{f2} are each independently selected from H and C₁-C₆ alkyl;
R^{x} and R^{y} are each independently selected from H and C₁-C₆ alkyl; or R^{x} and R^{y}, together with the nitrogen atom to which they are attached, form an optionally substituted 4- to 8-membered ring;
wherein each aryl or heteroaryl is unsubstituted or substituted independently selected from halo, hydroxyl, C₁-C₄ alkyl, C₁-C₄ alkoxy, C₁-C₄ hydroxyalkyl, —P(O)(OH)₂, —B(OH)₂, —COOR^{g2}, —CONR^{h2}Rⁱ², —SO₂NR^{j2}R^{k2}, and —SO₂OR^{l2}; and
wherein each alkyl or cycloalkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOR^{g2}, —PO₃H₂, —SO₂OR^{l2}, and —NR^{m2}Rⁿ²;
wherein each R^{g2}, R^{h2}, Rⁱ², R^{j2}, R^{k2}, R^{l2}, R^{m2}, and Rⁿ² is independently selected from H and methyl;
wherein the compound of formula (III) is:
B—X (III)
or a salt thereof, wherein:
B is aryl, heteroaryl, C₁-C₆ alkyl, aryl-C₁-C₄-alkyl-, aryl-C₂-C₄-alkoxy-, heteroaryl-C₁-C₄-alkyl-, and heteroaryl-C₂-C₄-alkoxy-; and
X is selected from —COOH, —PO₃H₂, —B(OH)₂, and —SO₃H;
wherein each aryl and heteroaryl is independently unsubstituted or substituted with 1 or more substituents independently selected from halo, C₁-C₄ alkyl, C₁-C₄ alkoxy, hydroxy, C₁-C₄ hydroxyalkyl, —NR^{a3}R^{b3}, —COOR^{c3}, —SO₃R^{d3}, —PO₃H₂, and —B(OH)₂;
wherein each alkyl is unsubstituted or substituted with 1 or more substituents independently selected from halo, C₁-C₄ alkoxy, hydroxy, thiol, C₁-C₄ alkylthio, optionally substituted C₃-C₆ cycloalkyl, optionally substituted 3- to 6-membered heterocyclyl, —COOH, —PO₃H₂ and —SO₃H; and
wherein R^{a3}, R^{b3}, R^{c3}, and R^{d3} are each independently selected from H, —CH₃, and —CH₂CH₃.

77.-84. (canceled)
